(19) United States
(12) Patent Application Publication
(10) Pub. No.: US 2025/0099455 A1
(43) Pub. Date: Mar. 27, 2025

(54) TREATMENT FOR CONGESTIVE HEART FAILURE

(71) Applicant: FibroGen, Inc., San Francisco, CA (US)

(72) Inventor: Ughetta Del Balzo, San Francisco, CA (US)

(21) Appl. No.: 18/728,812

(22) PCT Filed: Jan. 13, 2023
(86) PCT No.: PCT/US2023/060625

§ 371 Date: Jul. 12, 2024

Related U.S. Application Data
(60) Provisional application No. 63/299,383, filed on Jan. 13, 2022.
(60) Provisional application No. 63/306,994, filed on Feb. 4, 2022.

(51) Int. Cl. A61K 31/472; A61K 31/7048; A61P 9/04

(52) U.S. Cl. CPC A61K 31/472; A61K 31/7048; A61P 9/04

(57) ABSTRACT

The invention relates to treatment methods for improving cardiac function in subjects having congestive heart failure and particularly for preventing or reducing the decline in cardiac function that occurs subsequent to myocardial ischemia. The methods utilize compounds that selectively inhibit prolyl hydroxylase domain-containing protein 1 (PHD1). Some embodiments relate to combination therapies for treating heart failure with reduced ejection fraction (HFrEF) or heart failure with preserved ejection fraction (HFpEF).

### CROSS-REFERENCE TO RELATED APPLICATIONS

This application claims benefit of priority to U.S. Provisional Application No. 63/299,383, filed Jan. 13, 2022, and U.S. Provisional Application No. 63/306,994, filed Feb. 4, 2022, each of which is incorporated herein by reference in its entirety.

### BACKGROUND

### Field

The invention relates to the compound 3-[(1-cyano-4-hydroxy-7-phenoxy-isoquinoline-3-carbonyl)-amino-]-3-methyl butyric acid for use in treating heart failure, particularly congestive heart failure, in a patient in need thereof.

### State of the Art

Congestive heart failure (CHF) is a clinical syndrome defined as a chronic inadequate contraction of the heart muscle resulting in insufficient cardiac output. CHF may occur as a manifestation of an underlying condition, such as systemic or pulmonary hypertension; or subsequent to or in association with heart conditions such as myocardial infarction (MI), atherosclerosis, cardiomyopathy, congenital heart disease, or rheumatic fever. CHF affects more than 20 million people worldwide, with approximately 5.3 million Americans diagnosed with the syndrome. Prevalence of CHF follows an exponential curve rising with age and affecting 6-10% of people over age 65. Half of all patients diagnosed with CHF die within 4 years, and more than half of those diagnosed with severe CHF die within 1 year. An estimated 278,000 deaths related to CHF occur yearly in the United States. (See, e.g., Roger et al. (2011) Circulation 123:e18-e209; Remme et al. (2001) Eur Heart J 22:1527-1560; Stewart et al. (2001) Eur J Heart Fail 3:315-322.)

Current treatment of CHF usually involves lifestyle changes such as reducing salt in the diet and getting daily physical exercise. Medications used in CHF include angiotensin converting enzyme (ACE) inhibitors and angiotensin II receptor blockers (ARBs) to lower blood pressure; beta blockers to reduce heart rate; and diuretics to control fluid retention. Invasive treatments may also be used, including cardiac resynchronization therapy (CRT) in patients with wide QRS complexes on ECG; implantable cardioverter defibrillators (ICD) in patients with low ejection fraction; and heart transplantation in cases where all other treatments have failed to control symptoms.

Recent publications have suggested a role for hypoxia-inducible factor (HIF) in congestive heart disease. (See, e.g., Semenza (2000) Adv Exp Med Biol 475:123-130.) HIF is a heterodimeric nuclear protein (HIFα/β) that mediates the activation of cellular defense mechanisms during hypoxia. During conditions of normoxia, the HIF subunits are constitutively expressed, but the HIFα subunit is targeted for proteasome-mediated degradation by prolyl hydroxylation carried out by prolyl hydroxylase domain-containing proteins (PHD1, 2, and 3; also known as HPH (HIF prolyl hydroxylases)-3, 2, and 1 and EGLN-2, 1, and 3, respectively). (Fong and Takeda (2008) Cell Death and Differentiation. 15:635-641; Bernhardt et al. (2007) Methods in Enzymology. 435:221-245.)

Publications disclosing methods for treating CHF utilizing HIF contradictorily include both activating HIF, particularly by inhibiting PHD proteins (International Publication No. WO 2003/049686; Philipp et al. (2006) Eur J Heart Fail 8(4):347-354; Bao et al. (2010) J Cardiovasc Pharmacol 56(2):147-155); and inhibiting HIF (United States Patent Application Publication No. US 2010/008908.) In each publication, data is provided demonstrating changes in cardiac function associated with the methods disclosed therein. Support for each method relies on data provided by inhibiting PHD proteins, the former using an inhibitor of all three PHDs (a pan inhibitor) and the latter using a PHD3-specific knockout. However, research has shown that the three PHD isoforms function differently in their regulation of HIF and may have other non-HIF related regulatory roles.

A number of studies have been done to better define the roles of each of the PHD isoforms. Many of these studies were done using genetically engineered knockout or knockdown animals for each of the PHD genes, or using siRNA, shRNA, or RNAi specific for a single isoform to inhibit or reduce gene expression. For PHD1, studies have suggested that inhibition of this protein could be therapeutically beneficial for treating skeletal muscle cell degeneration (U.S. Pat. No. 7,858,593), for protection of myofibers against ischemia (Aragones et al. (2008) Nat. Genet. 40:170-180), for protecting against liver ischemia reperfusion (Schneider et al. (2010) Gastroenterology 138(3):1143-1154), and for treatment of colitis and other forms of inflammatory bowel disease (Tambuwala et al. (2010) Gastroenterology 139:2093-2101). For PHD2, studies have suggested contradictory roles in cardioprotection. In one study, somatic inactivation of PHD2 caused polycythemia and CHF; whereas in another study, cardiomyocyte-specific PHD2 knock-out improved cardiac function following myocardial infarction. (See Minamishima et al. (2008) Blood 111:3236-3244; and Holscher et al. (2011) J Biol Chem 286(13):11185-11194.)

### SUMMARY

The present inventors have now discovered that the compound 3-[(1-cyano-4-hydroxy-7-phenoxy-isoquinoline-3-carbonyl)-amino-]-3-methyl butyric acid, which selectively inhibits prolyl hydroxylase domain-containing protein 1 (PHD1), having the structure of Formula I

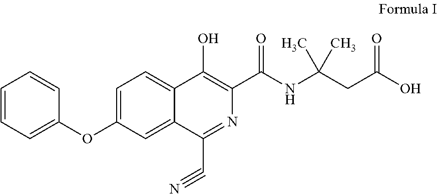 [SMILES: CC(C)(CC(=O)O)NC(=O)c1nc(C#N)c2cc(Oc3ccccc3)ccc2c1O]

is surprisingly effective in improving cardiac function in subjects having congestive heart failure and particularly in preventing or reducing the decline in cardiac function that occurs subsequent to myocardial ischemia. To be clear, 3-[(1-cyano-4-hydroxy-7-phenoxy-isoquinoline-3-carbonyl)-amino-]-3-methyl butyric acid is also referred to herein as the “compound of Formula I” or as “Formula I” or “Formula 1.” Experimental results in animal models suggest that use of the compound also surprisingly produces no significant increase in hematocrit or circulating hemoglobin levels, or in circulating erythropoietin levels, which property provides an advantage over the use of non-selective PHD inhibitors (that is, compounds that inhibit all three PHD isoforms) for treatment of CHF. Accordingly, the present invention provides, in one embodiment, a method of treating congestive heart failure in a subject in need thereof, said method comprising administering to the subject an effective amount of the compound of Formula I, thereby treating the congestive heart failure. The subject in need thereof is a subject having, or at risk of having, congestive heart failure.

In another embodiment, the invention provides a method for improving cardiac function in a subject in need thereof, said method comprising administering to said subject an effective amount of the compound of Formula I, thereby improving cardiac function. In various embodiments, the indicator of cardiac function may be selected from the group consisting of ejection fraction, stroke work, stroke volume, cardiac output, relaxation time, end systolic volume, end diastolic volume, and cardiac contractility.

In another embodiment, the invention provides a method for preventing or reducing decline in cardiac function in a subject in need thereof, said method comprising administering to said subject an effective amount of the compound of Formula I, thereby preventing or reducing the decline in cardiac function. In various embodiments, the indicator of cardiac function may be selected from the group consisting of ejection fraction, stroke work, stroke volume, cardiac output, relaxation time, end systolic volume, end diastolic volume, and cardiac contractility.

In another embodiment, the invention provides a method for reducing cardiac damage in a subject in need thereof, said method comprising administering to said subject an effective amount of a compound of Formula I, thereby reducing cardiac damage. In various embodiments, the cardiac damage may be selected from the group consisting of infarct size resulting from myocardial infarction, cardiac hypertrophy, and peri-infarct vessel formation.

In one embodiment, the compound of Formula I is administered to a subject having or at risk for having congestive heart failure. In a particular embodiment, the compound of Formula I is administered to a subject subsequent to a myocardial infarction in the subject. In other embodiments, the compound of Formula I is administered to a subject subsequent to a diagnosis of atherosclerosis, cardiomyopathy, congenital heart disease, or rheumatic fever in the subject. In one embodiment, the compound of Formula I is administered to prevent congestive heart failure following a myocardial ischemic event.

In various embodiments, the method comprises periodic administration of the compound of Formula I over an extended duration. The frequency of administration may be, e.g., once every 12, 24, 36, 48, 60, or 72 hrs, once a day, once every 2, 3, 4, 5, or 6 days, or once a week; and the duration of treatment may be one or more days, one or more weeks, one or more months, or one or more years. The methods can be used long-term, for improving cardiac function without producing concomitant hyperviscosity syndrome and volume overload due to polycythemia. The compound of Formula I is non-erythropoietic, making the compound an ideal candidate for treatment of chronic conditions, such as CHF, in which increased erythrogenesis is not desirable.

The invention also provides the compound of Formula I for use in treating congestive heart failure. In various embodiments, the invention provides the compound of Formula I for improving cardiac function, for preventing or reducing decline in cardiac function, and for preventing cardiac damage. The compound can be used long-term, improving cardiac function without concomitant hyperviscosity syndrome and volume overload associated with polycythemia.

In the embodiments provided above, the compound of Formula I may be used alone or in combination with an inhibitor of sodium-glucose cotransporter 2 (SGLT2). SGLT2 inhibitors, also called gliflozins, for use in the invention include, but are not limited to, canagliflozin, dapagliflozin, and empagliflozin.

In any embodiments of the methods, the compound of Formula I may be administered in combination with other medications, such as ACE inhibitors, ARBs, beta-blockers, and diuretics.

### BRIEF DESCRIPTION OF THE DRAWINGS

FIGS. 1A, 1B and 1C. The effect of the compound of Formula I at 6 and 20 mg/kg on cardiac function in an animal model of congestive heart failure subsequent to myocardial infarction as measured by a change in ejection fraction (FIG. 1A), relaxation time (FIG. 1B), and stroke work (FIG. 1C). Mean±SE, n=12; *p<0.05 vs vehicle; **p<0.01 vs vehicle; ***p<0.001 vs vehicle

FIGS. 2A and 2B. The effect of the compound of Formula I at 6 and 20 mg/kg on cardiac function due to delayed treatment of congestive heart failure subsequent to myocardial infarction in an animal model as measured by a change in ejection fraction (FIG. 2A) and end-systolic volume (FIG. 2B). Mean±SE, n=11-12; *p<0.05 vs vehicle; **p<0.01 vs vehicle; ***p<0.001 vs vehicle; ###p<0.001 vs Baseline.

FIGS. 3A and 3B. Long-term treatment with the compound of Formula I at 6 and 20 mg/kg produced no erythropoietic effect in the animal model of congestive heart failure subsequent to myocardial infarction as measured by change in hemoglobin (FIG. 3A) and hematocrit (FIG. 3B). Mean±SE, n=11-12.

FIGS. 4A, 4B and 4C. The effect of daily dosing with the compound of Formula I at 2, 6, and 20 mg/kg on cardiac function in an animal model of congestive heart failure subsequent to myocardial infarction as measured by a change in ejection fraction (FIG. 4A), end-systolic volume (FIG. 4B), and stroke work (FIG. 4C). Mean±SE, n=12; *p<0.05 vs vehicle; **p<0.01 vs vehicle; ***p<0.001 vs vehicle.

FIG. 5 is a graph depicting the dose-dependent reducing affect of compound of Formula I on infarct size post-acute myocardial ischemia.

FIG. 6A is a graph showing the compound of Formula I improved left ventricular function (LVDP) after global ischemia.

FIG. 6B is a graph showing the compound of Formula I improved left ventricular function (LVEP) after global ischemia

FIG. 6C is a graph showing the compound of Formula I improved coronary flow after global ischemia.

FIG. 7A shows a graph showing the pressure volume relationship.

FIG. 7B shows a graph depicting heart weight/body weight ratios.

FIG. 8A is a graph showing ejection fraction after 28 days of dosing (QOD).

FIG. 8B is a graph showing stroke work after 28 days of dosing (QOD).

FIG. 8C is a graph showing stroke volume after 28 days of dosing (QOD).

FIG. 8D is a graph showing cardiac output.

FIG. 9 is a graph depicting cardia histology, 28 days of dosing (QOD).

FIGS. 10A and 10B depict dose response with the compound of Formula I on cardiac function in congestive heart failure (CHF) was studied by inducing CHF following myocardial ischemia for the determination of Minimum Effective Dose (MED) 2 mg/kg, as shown in FIGS. 10A and 10B.

FIGS. 11A and 11B depict the effect of duration of administration of the compound of Formula I on cardiac function in congestive heart failure (CHF) by inducing CHF subsequent to myocardial infarction.

FIG. 11C is a graph depicting effect on the collagen area.

FIG. 12 is a graph depicting ejection fraction, evidencing developing heart failure with a preserved ejection fraction.

FIG. 13A is a graph depicting improvement in diastolic function.

FIG. 13B is a graph depicting improvement in left atrium weight.

FIG. 13C is a graph depicting improvement in relaxation time.

FIG. 14D is a graph depicting improvement in dP/dt minimum.

FIG. 14A is a graph depicting mean blood pressure,

FIG. 14B is a graph depicting diastolic blood pressure.

FIG. 14C is a graph depicting systolic blood pressure.

FIG. 15A is a graph depicting ejection fraction of monotherapies of the compound of Formula I and an exemplary SGLT2i, empagliflozin.

FIG. 15B is a graph depicting end systolic volume for monotherapies of the compound of Formula I and an exemplary SGLT2i, empagliflozin.

FIG. 15C is a graph depicting end diastolic volume the compound of Formula I and an exemplary SGLT2i, empagliflozin.

FIG. 16A is a graph depicting ejection fraction of the compound of Formula I plus empagliflozin in combination compared to monotherapies.

FIG. 16B is a graph depicting end systolic volume of the compound of Formula I plus empagliflozin in combination compared to monotherapies.

FIG. 17A is a graph depicting the coronary perfusion pressure after treatment with the compound of Formula.

FIG. 17B is a graph depicting the coronary resistance after treatment with the compound of Formula.

FIG. 18 depicts the PK profile of the Compound of Formula I in rats (high oral bioavailability & low CL).

FIG. 19 depicts the preliminary PK profile of the compound of Formula I in dogs.

FIG. 20 depicts the PK profile of the compound of Formula I in monkeys.

FIG. 21 depicts LV relaxation after treatment with the compound of Formula I and an exemplary SGLT2i, empagliflozin, compared to monotherapies

FIG. 22 depicts LV minimum pressure after treatment with the compound of Formula I and an exemplary SGLT2i, empagliflozin, compared to monotherapies.

FIG. 23 depicts end diastolic pressure after treatment with the compound of Formula I and an exemplary SGLT2i, empagliflozin, compared to monotherapies.

FIGS. 24A-C depict graphs showing LV relaxation time, LV minimum pressure, and end-diastolic pressure demonstrating the combination of the compound of Formula I plus empagliflozin compared to monotherapies showed an additive effect in the treatment of HFpEF with improvement in left ventricle filling pressure (minimum and end-diastolic pressures).

### DETAILED DESCRIPTION

Before the present compositions and methods are described, it is to be understood that the invention is not limited to the particular compounds, compositions, methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein, and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention.

The practice of the present invention will employ, unless otherwise indicated, conventional methods of chemistry, biochemistry, molecular biology, cell biology, genetics, immunology, and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; D. M. Weir, and C. C. Blackwell, eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. 1-111, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton & Graham eds. (1997) PCR (Introduction to Biotechniques Series), 2nd ed., Springer Verlag).

The terms “PHD”, “prolyl hydroxylase domain-containing protein”, “HIF prolyl hydroxylase,” “HPH,” and “HIF PH” refer to members of the Egl-Nine (EGLN) gene family described by Taylor (2001, Gene 275:125-132), and characterized by Aravind and Koonin (2001, Genome Biol 2: RESEARCH 0007), Epstein et al. (2001, Cell 107:43-54), and Bruick and McKnight (2001, Science 294:1337-1340). The term “PHD1” refers to prolyl hydroxylase domain-containing protein-1. This protein is also sometimes referred to as EIT6, HIF-PH1, HIFPH1, EGLN2 and HPH-3. PHD1 proteins include, but are not limited to, human PHD1 (GenBank Accession No. NP_444274.1), human EGLN2 isoform 1 (GenBank Accession No. CAC42510; Taylor, supra), human EGLN2 isoform 3 (GenBank Accession No. NP_542770), mouse EGLN2 (GenBank Accession No. CAC42516), and rat EGLN2 (GenBank Accession No. AA046039). PHD2 (also known as EGLN-1) include, but are not limited to, human EGLN1 (GenBank Accession No. AAG33965; Dupuy et al. (2000) Genomics 69:348-54), mouse EGLN1 (GenBank Accession No. CAC42515), and rat EGLN1 (GenBank Accession No. P59722). PHD3 (also known as EGLN-3) include, but are not limited to, human EGLN3 (GenBank Accession No. CAC42511; Taylor, supra), mouse EGLN3 (GenBank Accession No. CAC42517), and rat EGLN3 (SM-20) (GenBank Accession No. AAA19321). In other embodiments of the present invention, EGLN may include Caenorhabditis elegans EGL-9 (GenBank Accession No. AAD56365) and Drosophila melanogaster CG1114 gene product (GenBank Accession No. AAF52050). All GenBank sequences associated with the provided accession Nos. are herein incorporated by reference in their entirety.

In addition to those provided above for PHD1 protein, a further GenBank Accession No. associated with the human PHD1 gene is NM_053046.3 (mRNA), which is herein incorporated by reference in its entirety.

The terms “disorders,” “diseases,” and “conditions” are used inclusively herein and refer to any condition deviating from normal.

The terms “treating,” “treatment” and the like, are used herein to mean administering a therapy to a patient in need thereof.

An “effective amount” of a compound is an amount sufficient to bring about the desired result in a treated subject, for example, an amount sufficient to treat congestive heart failure, to improve cardiac function, to prevent or reduce decline in cardiac function, or to reduce cardiac damage. The effective amount can vary depending upon the particular compound, the nature or severity of the condition being treated, the age, weight, etc. of the subject being treated, the route of administration or formulation of the compound, and the dosing regimen, among other things. An effective amount can readily be determined by one skilled in the medical arts.

The term “ester” refers to compounds of Formula I wherein the carboxylic acid is in the form —COOR where R is alkyl, substituted alkyl, aryl, or substituted aryl. For example, esters Formula I include compounds wherein R is alkyl. Esters of Formula I can be provided, for example, via esterification of the hydroxyl group of the carboxylic acid using a suitable reagent such as an acylhalide or an anhydride and/or via esterification of the carboxylic acid moiety. Such methods are well known in the art.

The term “pharmaceutically acceptable salt” refers to pharmaceutically acceptable salts of Formula I, which salts are derived from a variety of organic and inorganic counter ions well known in the art, and include, by way of example only, sodium, potassium, calcium, magnesium, ammonium, tetraalkylammonium, and the like. Pharmaceutically acceptable salts of the invention can be provided by compounds of Formula I at the hydroxyl group at the C4 position of the isoquinoline, and/or at the carboxylic acid moiety by methods well known in the art.

The terms “stereoisomer” or “stereoisomers” refer to compounds that differ in the chirality of one or more stereocenters. Stereoisomers include enantiomers (compounds are non-superimposable mirror images) and diastereomers (compounds having more than one stereogenic center that are non-mirror images of each other and wherein one or more stereogenic center differs between the two stereoisomers). The compounds of the invention can be present as a mixture of stereoisomers or as a single stereoisomer.

The term “tautomer” refers to alternate forms of a compound that differ in the position of a proton, such as enol, keto, and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

The term “prodrug” as used herein, refers to compounds of Formula I that include chemical groups which, in vivo, can be converted into the carboxylate group and/or can be split off from the amide N-atom and/or can be split off from the C4 hydroxy to provide for the active drug, a pharmaceutically acceptable salt thereof, or a biologically active metabolite thereof. Suitable groups are well known in the art and particularly include: for the carboxylic acid moiety, a prodrug selected from, e.g., esters including, but not limited to, those derived from alkyl alcohols, substituted alkyl alcohols, hydroxy substituted aryls and heteroaryls and the like.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

The term “selectively” as used herein with respect to the ability of the compound of Formula I to inhibit PHD1 is intended to refer to a compound that is at least about five times more potent for inhibiting the activity of PHD1 than for inhibiting the activity of PHD2 and/or PHD3. Such compounds are referred to interchangeably herein as “PHD1 selective inhibitors,” “PHD1 selective inhibitor compounds,” and “compounds that selectively inhibit PHD1.” “Inhibit PHD1” or “inhibiting the activity of PHD1” means reducing, eliminating or attenuating the enzymatic activity of PHD1 protein. The selectivity of the compound can be determined by determining the IC₅₀ of the compound for each of the PHD isoforms. A compound that selectively inhibits PHD1 activity will be a compound exhibiting a lower IC₅₀ for PHD1 than for PHD2 and/or PHD3.

The term “alkyl” refers to saturated monovalent hydrocarbyl groups having from 1 to 10 carbon atoms, more particularly from 1 to 5 carbon atoms, and even more particularly 1 to 3 carbon atoms. This term is exemplified by groups such as methyl, ethyl, n-propyl, iso-propyl, n-butyl, t-butyl, n-pentyl, and the like. The term “C₁-C₄ alkyl” refers to an alkyl having from 1 to 4 carbon atoms and includes methyl, ethyl, n-propyl, iso-propyl, n-butyl, t-butyl, and the like.

The term “aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

The term “carboxyl” refers to —COOH or salts thereof.

The term “carboxyl ester” refers to the groups —C(O)O-alkyl, C(O)O-alkenyl, —C(O)O— alkynyl, —C(O)O-cycloalkyl, —C(O)O-aryl, —C(O)O-heteroaryl, and —C(O)O-heterocyclic.

The term “cyano” refers to the group —CN.

The term “hydroxy” or “hydroxyl” refers to the group —OH.

The term “heteroaryl” refers to an aromatic ring of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, or thienyl) or multiple condensed rings (e.g., indolizinyl or benzothienyl) provided the point of attachment is through a ring containing the heteroatom and that ring is aromatic. The nitrogen and/or sulfur ring atoms can optionally be oxidized to provide for the N-oxide or the sulfoxide, and sulfone derivatives. Examples of heteroaryls include but are not limited to, pyridinyl, pyrimidinyl, pyrrolyl, pyrazolyl, indolyl, thiophenyl, thienyl, and furyl.

### Parameters of Cardiac Function

End-diastolic volume (EDV) is the volume of blood in a ventricle at the end of filling, or diastole; and end-systolic volume (ESV) is the volume of blood in the ventricle at the end of contraction, or systole. ESV is a clinical measure of the adequacy of cardiac emptying. ESV can be measured using 2-dimensional echocardiography, magnetic resonance imaging (MRI), or computerized tomography (CT). The stroke volume (SV) is the output of blood by the heart during a single phase of the cardiac cycle and is calculated by subtracting the ESV from the EDV.

Ejection fraction (EF) refers to the percentage of blood pumped out of a filled ventricle with each heartbeat. Ejection fraction is typically measured in the left ventricle (LV) and is normally in the range of 55 to 70%. A reduction in EF indicates weakness in the heart muscle, and may result when heart muscle has become damaged due to a heart attack, heart muscle disease (cardiomyopathy), or other causes. An EF of less than 40% is indicative of heart failure, and an EF of less than 35% increases the risk of sudden cardiac arrest. EF can be calculated by dividing the SV by the EDV, and can be measured with imaging techniques including echocardiogram, cardiac catheterization, MRI, CT, and nuclear medicine scan (multiple gated acquisition (MUGA); also called a nuclear stress test) of the heart.

Cardiac output (CO) refers to the volume of blood expelled by the ventricles per minute. A normal, resting adult has a cardiac output of 4 to 8 liters per minute. Cardiac output can be measured using the thermodilution technique, or can be calculated by multiplying the SV by the heart rate.

Stroke work (SW) refers to the work done by the ventricle in ejecting a volume of blood into the aorta. Stroke work can be estimated by multiplying the SV by the mean aortic pressure (MAP).

Cardiac contractility is the inotropic state of the myocardium, and is a major determinant of cardiac output and an important factor in cardiac compensation. The maximum rate of rise of left ventricular pressure (dP/dtₘₐₓ) is an index of the initial velocity of myocardial contraction, and is used clinically to characterize the contractile ability of the heart. The normal maximum left ventricular dP/dt is about 1600 mm Hg/sec, but is often less than 1200 mm Hg/sec in patients with disorders of the myocardium. Relaxation time, as measured by the left ventricular relaxation time constant (Tau), is used to evaluate left ventricular diastolic function, and is a sensitive sign of left ventricular dysfunction during the development of chronic heart failure. The normal range is 45-85 msec, but can be prolonged to >90 msec in various diseases that result in impairment of myocardial relaxation. Tau can be calculated by dividing the left ventricular pressure (P) by −dP/dt. End systolic pressure volume relationship (ESPVR) describes the maximal pressure that can be developed by the ventricle at any given LV volume. The slope of ESPVR represents the end-systolic elastance, which provides an index of myocardial contractility. The ESPVR is relatively insensitive to changes in preload, afterload and heart rate. This makes it an improved index of systolic function over other hemodynamic parameters like ejection fraction, cardiac output and stroke volume. The ESPVR becomes steeper and shifts to the left as inotropy (contractility) increases. The ESPVR becomes flatter and shifts to the right as inotropy decreases.

Damage to the myocardium can result from an acute assault, such as a myocardial infarction, or from a chronic assault, such as the chronic strain placed on the heart due to high blood pressure or atherosclerosis. Chronic damage can and often does occur subsequent to a myocardial infarction as well, due to the reduced pumping capacity of the damaged heart. Under physical load heart muscle can thicken (hypertrophy), resulting in a decrease in the size of the chamber of the heart. Healthy cardiac hypertrophy (physiologic hypertrophy) is the normal response to healthy exercise, which results in an increase in the heart's muscle mass and pumping ability. Unhealthy cardiac hypertrophy (pathological hypertrophy) is the response to stress or disease, which leads to an enlargement of myocardial cells, hyperplasia of nonmuscular cardiac components, and decreased pumping ability. In pathological hypertrophy, the mass of the heart can increase by up to 150%. As used herein, “cardiac hypertrophy” refers to pathological hypertrophy.

### Methods

The methods of the invention employ the compound of Formula I

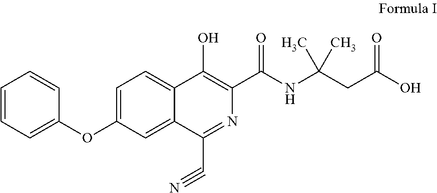 [SMILES: CC(C)(CC(=O)O)NC(=O)c1nc(C#N)c2cc(Oc3ccccc3)ccc2c1O]

which selectively inhibits PHD1 over other PHD isoforms, for example, over PHD2 and/or PHD3. The present inventors have found that, advantageously, the compound of Formula I can have the beneficial therapeutic effect of improving cardiac function in subjects having congestive heart failure without concomitantly producing side effects resulting from the inhibition of the other PHD isoforms. In particular, the compound of Formula I does not significantly increase circulating erythropoietin, hemoglobin levels, or hematocrit in treated subjects. Therefore, the methods have the substantial benefit that they can be used long-term to improve cardiac function without cardiac complications that are associated with hyperviscosity syndrome and volume overload resulting from polycythemia.

In one embodiment, the present invention provides a method of treating congestive heart failure in a subject in need thereof, said method comprising administering to the subject an effective amount of the compound of Formula I, thereby treating the congestive heart failure. Treating congestive heart failure may encompass, inter alia, improving cardiac function (for example, as determined by improvement, or positive change, in one or more well known parameters of cardiac function), preventing or reducing a decline in cardiac function (for example, as determined by prevention or reduction of a decline, or lack of a significant negative change, in one or more well known parameters of cardiac function), or reducing cardiac damage (for example, as determined by the effect on one or more well known markers of cardiac damage). Although some examples of parameters of cardiac function and markers of cardiac damage are described herein, there are many more that are well known and appropriate for use in assessing the efficacy of the treatment, as would be readily recognized by a competent medical practitioner.

In another embodiment, the invention provides a method for improving cardiac function in a subject in need thereof, said method comprising administering to said subject an effective amount of the compound of Formula I, thereby improving cardiac function. In various embodiments, the indicator of cardiac function may be selected from the group consisting of ejection fraction, stroke work, stroke volume, cardiac output, relaxation time, end systolic volume, end diastolic volume, and cardiac contractility. In some embodiments, the indicator of cardiac function may be selected from the group consisting of ejection fraction, stroke work, stroke volume, and cardiac output.

In another embodiment, the invention provides a method for preventing or reducing decline in cardiac function in a subject in need thereof, said method comprising administering to said subject an effective amount of the compound of Formula I, thereby preventing or reducing the decline in cardiac function. In various embodiments, the indicator of cardiac function may be selected from the group consisting of ejection fraction, stroke work, stroke volume, cardiac output, relaxation time, end systolic volume, end diastolic volume, and cardiac contractility. In some embodiments, the indicator of cardiac function may be selected from the group consisting of ejection fraction, stroke work, stroke volume, and cardiac output.

Cardiac function can be determined by any method generally known to those of skill in the art. The methods of the invention can be administered to a subject whenever the subject's cardiac function is outside of the normal range, typically below or significantly below normal range such as when any one or more of EDV, ESV, SV, EF, CO, and SW are below the typical value or normal range. It is within the competence of medical practicioners of ordinary skill to determine whether a subject has cardiac function that is outside of the normal range to an extent that treatment with the methods and compounds described herein would be beneficial. For example, the normal range of left ventricular cardiac function in humans for parameters listed herein are as follows:

| Measure | Normal range | Typical value |
|---|---|---|
| End-diastolic volume (EDV) | 65-240 mL | 120 mL |
| End-systolic volume (ESV) | 16-143 mL | 50 mL |
| Stroke Volume (SV) | 55-100 mL | 70 mL |
| Ejection Fraction (EF) | 55-70% | 58% |
| Cardiac Output (CO) | 4.0-8.0 L/min | 5.25 L/minute |
| Stroke Work (SW) | 58-104 gm-m/beat | 76 gm-m/beat |

In another embodiment, the invention provides a method for reducing cardiac damage in a subject in need thereof, said method comprising administering to said subject an effective amount of the compound of Formula I, thereby reducing cardiac damage. In various embodiments, the cardiac damage may be selected from the group consisting of infarct size (resulting from a myocardial infarction), cardiac hypertrophy (as measured, e.g., by cardiomyocyte diameter), and peri-infarct vessel formation.

In one embodiment, the compound of Formula I is administered to a subject having or at risk for having congestive heart failure. In a particular embodiment, the compound is administered to a subject subsequent to a myocardial infarction in the subject. In one embodiment, the compound is administered to prevent congestive heart failure following a myocardial ischemic event in the subject. In other embodiments, the compound is administered to a subject subsequent to a diagnosis of atherosclerosis, cardiomyopathy, congenital heart disease, or rheumatic fever in the subject.

In various embodiments, the method comprises periodic administration of the compound of Formula I over an extended duration. The frequency of administration in the periodic administration may be, e.g., once every 12, 24, 36, 48, 60, or 72 hrs, once a day, once every 2, 3, 4, 5, or 6 days, or once a week; and the duration of treatment may be one or more days, one or more weeks, one or more months, or one or more years. An extended duration of treatment typically is more than one administration of compound.

The methods can advantageously be used long-term, improving cardiac function without concomitant hyperviscosity syndrome and volume overload due to polycythemia. In certain embodiments is provided a method for treating congestive heart failure in a subject in need thereof comprising administering to the subject an effective amount of the compound of Formula I, wherein the administration of the compound does not produce a concomitant significant increase in circulating hemoglobin level in the subject. In certain embodiments is provided a method for treating congestive heart failure in a subject in need thereof comprising administering to the subject an effective amount of the compound of Formula I, wherein the administration of the compound does not produce a concomitant significant increase in circulating erythropoietin level in the subject. In certain embodiments is provided a method for treating congestive heart failure in a subject in need thereof comprising administering to the subject an effective amount of the compound of Formula I, wherein the administration of the compound does not produce a concomitant significant increase in hematocrit in the subject.

### Subjects

In various embodiments, the subject is an animal, particularly an animal of mammalian species including rat, rabbit, bovine, ovine, porcine, murine, equine, and primate species. In a most preferred embodiment, the subject is a human.

In general, a suitable subject for practice of the method of the present invention (that is, a subject in need of treatment for congestive heart failure, a subject in need of improving cardiac function, a subject in need of preventing or reducing a decline in cardiac function, or a subject in need of reducing cardiac damage) includes any subject having, or at risk for having, congestive heart failure. Suitable subjects include any mammalian subjects, in particular, human subjects. In various embodiments, the subject has congestive heart failure. In particular embodiments, the subject has, or is at risk for having, congestive heart failure due to myocardial infarction in the subject. In other embodiments, the subject has, or is at risk for having, congestive heart failure as a result of cardiac damage. In these embodiments, the cardiac damage can be selected from the group consisting of infarct size, cardiac hypertrophy, and peri-infract vessel formation. In other embodiments, the subject has, or is at risk for having, congestive heart failure due to coronary artery disease, hypertension, atherosclerosis, cardiomyopathy, congenital heart disease, or rheumatic fever.

In general, a suitable subject for practice of the method of the present invention includes any subject having reduced cardiac function. In various embodiments, the indicator of cardiac function may be selected from the group consisting of ejection fraction, stroke work, stroke volume, cardiac output, relaxation time, and cardiac contractility.

### Compounds

The methods of the present invention utilize the compound of Formula I

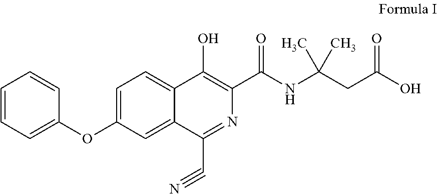 [SMILES: CC(C)(CC(=O)O)NC(=O)c1nc(C#N)c2cc(Oc3ccccc3)ccc2c1O]

which is capable of inhibiting PHD1 selectively over other isoforms, for example, PHD2 and/or PHD3 enzymes. As a result of this selectivity profile, the compound is typically non-erythropoietic at the doses required for practice of the present invention, making the compound an ideal candidate for chronic conditions such as congestive heart failure in which increased erythrogenesis is not desired.

Recitation of “the compound of Formula I” as used herein is generally not limited to the free acid form shown in Formula I, but includes pharmaceutically acceptable salts, esters, tautomers, and prodrugs of the compound as well where use of such forms is appropriate or preferred.

### HIF-PH Assay

The selectivity profile for the compound of Formula I can be determined using, for example, any assay familiar and commonly accessible to those skilled in the art, e.g., the following HIF-PH assay.

Ketoglutaric acid α-[1-¹⁴C]-sodium salt, alpha-ketoglutaric acid sodium salt, and HPLC purified peptide may be obtained from commercial sources, e.g., Perkin-Elmer (Wellesley MA), Sigma-Aldrich, and SynPep Corp. (Dublin CA), respectively. Peptides for use in the assay may be fragments of HIFα as described above or as disclosed in International Publication WO 2005/118836, incorporated by reference herein. HIF-PH, e.g., PHD1 (EGLN2) or PHD2 (EGLN1), can be expressed in, e.g., insect Hi5 cells, and partially purified, e.g., through a SP ion exchange chromatography column. Enzyme activity is determined by capturing ¹⁴CO₂ using an assay described by Kivirikko and Myllyla (1982, Methods Enzymol. 82:245-304). Assay reactions contain 50 mM HEPES (pH 7.4), 100 μM α-ketoglutaric acid sodium salt, 0.30 μCi/mL ketoglutaric acid α-[1-¹⁴C]-sodium salt, 40 μM FeSO₄, 1 mM ascorbate, 1541.8 units/mL Catalase, with or without 50 M peptide substrate and various concentrations of compound of the invention. Reactions are initiated by addition of HIF-PH enzyme. IC₅₀ (compound concentration for 50% inhibition of enzyme activity) of the compounds for each enzyme can be determined.

The peptide-dependent percent turnover is calculated by subtracting percent turnover in the absence of peptide from percent turnover in the presence of substrate peptide. Percent inhibition and IC₅₀ are calculated using peptide-dependent percent turnover at given inhibitor concentrations. Calculation of IC₅₀ values for each inhibitor is conducted using GraFit software (Erithacus Software Ltd., Surrey UK). IC₅₀ of test compound for PHD1 and for PHD2 and/or PHD3 can be readily determined using the above method, and used to assess the selectivity of the compound for inhibition of PHD1.

The compound of Formula I is at least about five times more active in inhibiting PHD1 enzyme over PHD2 enzyme; that is, the ratio of the IC₅₀ for PHD2 over the IC₅₀ for PHD1 (i.e., IC₅₀ PHD2/IC₅₀ PHD1) is greater than or equal to about five. By way of example, the selectivity for the compound of Formula I is shown below in Table 1. The selectivity for the compound of Formula I as demonstrated in an additional experiment is shown below in Table 1A.

TABLE 1

| Compound | IC₅₀ (μM) PHD1 | IC₅₀ (μM) PHD2 | PHD2/PHD1 |
|---|---|---|---|
| Formula I | 0.91 | 20.41 | 23 |

TABLE 1A

| Compound | IC₅₀ (μM) PHD1 | IC₅₀ (μM) PHD2 | PHD2/PHD1 |
|---|---|---|---|
| Formula I | 0.7 | 16.9 | 24 |

The compounds for use in the present methods are advantageous over compounds previously disclosed in the art. In particular, the use of a PHD1 selective inhibitor compound in the treatment of congestive heart failure improves cardiac function without significantly increasing hematocrit or hemoglobin. Congestive heart failure associated with somatic PHD2 inactivation was attributed to hyperviscosity syndrome and volume overload due to polycythemia. (Minamishima et al. (2008) Blood 111:3236-3244.) Use of a non-specific HIF prolyl hydroxylase inhibitor, while showing improvement in heart function, also showed increase in circulating erythropoietin that would limit its usefulness for long-term therapy due to resulting polycythemia. (Bao et al. (2010) J Cardiovasc Pharmacol 56(2):147-155.) Thus, the present invention provides a substantial benefit over the methods currently available in the art.

### Pharmaceutical Formulations and Routes of Administration

The compositions of the present invention can be delivered directly or in pharmaceutical compositions or medicaments along with suitable carriers or excipients, as is well known in the art. Present methods of treatment can comprise administration of an effective amount of a compound of the invention to a subject in need. In a preferred embodiment, the subject is a mammalian subject, and in a most preferred embodiment, the subject is a human subject.

A effective amount of such compound, composition, or medicament can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott NY; etc.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art.

For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the compounds of the invention are well-known in the art and include water-based solutions containing a base, such as, for example, sodium hydroxide, to form an ionized compound; sucrose or sodium chloride as a tonicity agent; and a buffer, for example, a buffer that contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective amount can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays. In certain embodiments, a compound of the disclosure is formulated for oral administration. An exemplary dose of a compound of the disclosure in a pharmaceutical formulation for oral administration is from about 0.01 mg/kg to about 60 mg/kg body weight of the subject, particularly from 0.5 to about 20 mg/kg body weight of subject. In some embodiments, a pharmaceutical formulation comprises from about 0.7 to about 6.0 mg/kg body weight of subject, or alternatively, from about 1.0 to about 3.0 mg/kg body weight of subject. A typical dosing regimen for oral administration would be administration of the pharmaceutical formulation for oral administration one time every 12, 24, 48, or 72 hours, once a day, once every 2, 3, 4, 5, or 6 days, or once a week.

An effective amount or a therapeutically effective amount or dose of an agent, e.g., a compound of the invention, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician.

Dosages particularly fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of compound or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack; or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein and are specifically contemplated.

### Examples

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention.

### Example 1. Effect of the Compound of Formula I on Cardiac Function in an Animal Model of Congestive Heart Failure

The effect of the compound of Formula I on cardiac function in congestive heart failure (CHF) was studied by inducing CHF subsequent to myocardial infarction (MI). Three groups of rats (n=12) were subjected to MI by 30 minute ligation of the left coronary artery followed by reperfusion. In a sham group, rats underwent a similar surgical procedure except that the left coronary artery was not occluded. Treatment with the compound of Formula I was initiated 2 days after reperfusion. In the MI groups, rats were dosed orally every other day with vehicle, 6 mg/kg or 20 mg/kg compound from day 2 to day 26 post MI. In the sham group, rats were dosed with vehicle on the same schedule. At 28 days post-MI, cardiac function, heart weight, and complete blood count (CBC) were measured. Histomorphometric analysis was performed on cardiac sections to quantify collagen deposition with picrosirius red staining (PSR), remodeling with hematoxylin and eosin staining and blood vessel number with immunohistochemistry for smooth muscle α-actin.

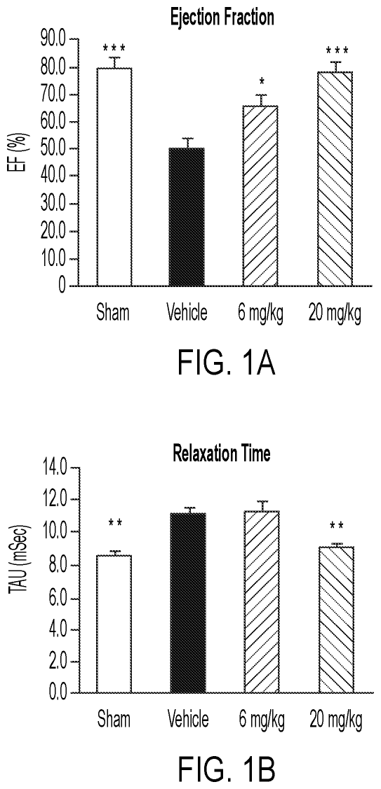

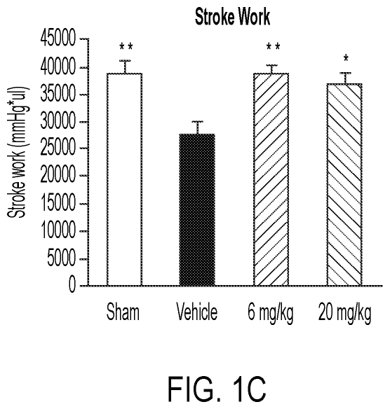

As shown in FIG. 1, treatment with the compound of Formula I significantly improved ejection fraction (FIG. 1A), relaxation time (FIG. 1B), and stroke work (FIG. 1C) compared to the vehicle-treated MI animals. Treatment with the compound of Formula I also significantly improved cardiac hypertrophy, stroke volume, cardiac output, end-systolic volume, end-systolic pressure, end-diastolic pressure, and arterial elastance compared with vehicle treated animals. Treatment with the compound of Formula I also normalized ESPVR slope. Histomorphometric analysis of the left ventricle showed prevention of ventricular wall thinning and significant reduction in hypervascularization in the peri-infarct area. Treatment with the compound of Formula I did not produce a concomitant significant increase in circulating hemoglobin level or have a significant effect on other CBC parameters.

A similar experiment was carried out in which rats were dosed more frequently with a lower dose. Animals were dosed orally every day with vehicle, 2 mg/kg, 6 mg/kg or 20 mg/kg compound from day 2 to day 26 post MI. These results demonstrate that providing a lower dose at more frequent intervals is equally effective as a higher dose at less frequent intervals.

These results indicate that administration of the compound of Formula I effectively treats congestive heart failure. Administering the compound of Formula I every day or every other day for 28 days significantly improved cardiac function in congestive heart failure following MI, including significant improvement in ejection fraction, stroke work, stroke volume, cardiac output, relaxation time, cardiac hypertrophy, end-systolic volume, and cardiac contractility. Improvement in cardiac function and treatment of congestive heart failure is achieved without any concomitant rise in blood hemoglobin.

### Example 2. Effect of Delayed Treatment with the Compound of Formula I on Cardiac Function in an Animal Model of Congestive Heart Failure

The effect of delayed treatment with the compound of Formula I on cardiac function in congestive heart failure (CHF) was studied by inducing CHF subsequent to MI. Four groups of rats (n=11-12) were subjected to MI by 30 minute ligation of the left coronary artery followed by reperfusion. In a sham group, rats underwent a similar surgical procedure except that the left coronary artery was not occluded. Cardiac function was measured in one group at 28 days post-MI (Baseline). In the remaining MI groups, dosing with vehicle or test article was initiated on Day 28. Rats were orally dosed every other day with vehicle, 6 mg/kg or 20 mg/kg compound from day 28 until day 54 post-MI. At 56 days post-MI, cardiac function, heart weight and complete blood count (CBC) were measured.

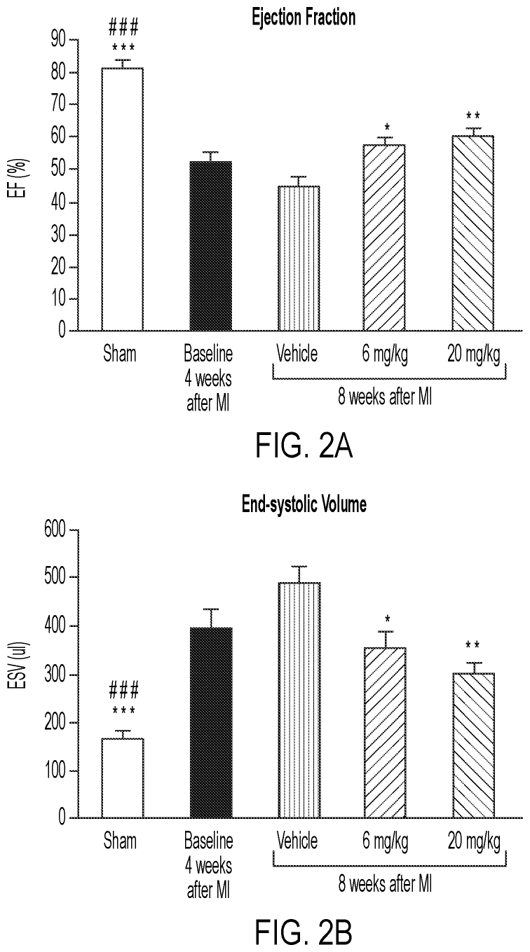

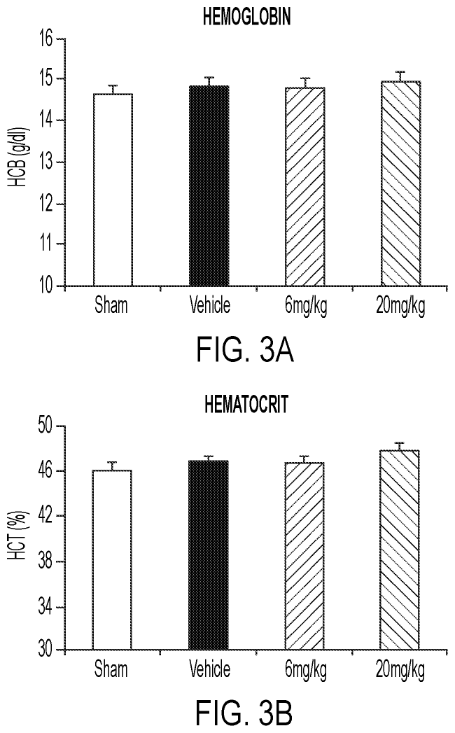

As shown in FIG. 2, delayed treatment with the compound of Formula I significantly improved ejection fraction (FIG. 2A) and end-systolic volume (FIG. 2B) compared with vehicle-treated MI animals at 56 days post-MI. Delayed treatment with the compound of Formula I also improved cardiac hypertrophy, dPdtₘₐₓ, relaxation time, and end-diastolic volume. Treatment with the compound of Formula I had no significant effect on hematocrit (FIG. 3A) or hemoglobin level (FIG. 3B), demonstrating that long-term treatment of congestive heart failure using the compound of Formula I does not lead to complicating hyperviscosity syndrome and volume overload due to polycythemia.

These results indicate that administration of the compound of Formula I effectively improves cardiac function when administered at more advanced stages of congestive heart failure. Initiating administration of the compound of Formula I 28 days after MI, and administering every other day for 28 days significantly improved cardiac function, including improvement in ejection fraction, end-systolic volume, relaxation time, cardiac hypertrophy, and cardiac contractility.

### Example 4. Effect of the Compound of Formula I on Cardiac Function in an Animal Model of Congestive Heart Failure—Daily Dosing

The effect of the compound of Formula I on cardiac function in congestive heart failure (CHF) was studied by inducing CHF subsequent to myocardial infarction (MI). Four groups of rats (n=12) were subjected to MI by 30 minute ligation of the left coronary artery followed by reperfusion. In a sham group, rats underwent a similar surgical procedure except that the left coronary artery was not occluded. Treatment with the compound of Formula I was initiated 2 days after reperfusion. In the MI groups, rats were dosed orally daily with vehicle, 2 mg/kg, 6 mg/kg or 20 mg/kg compound from day 2 to day 26 post MI. In the sham group, rats were dosed with vehicle on the same schedule. At 28 days post-MI, cardiac function, heart weight, and complete blood count (CBC) were measured.

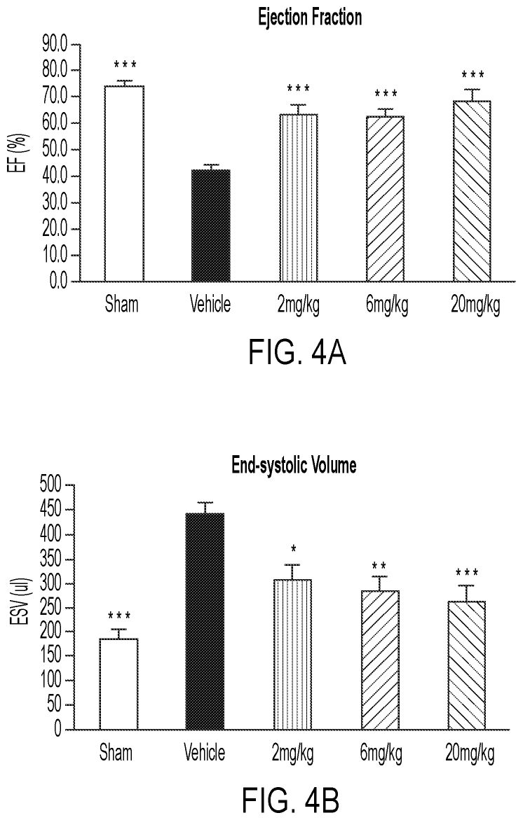

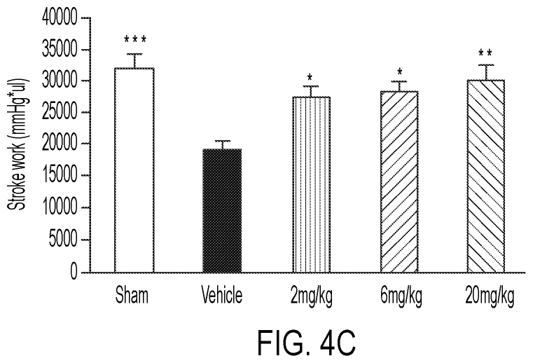

Daily treatment with the compound of Formula I significantly improved ejection fraction (FIG. 4A), end systolic volume (FIG. 4B), and stroke work (FIG. 4C) compared to the vehicle-treated MI animals. Daily treatment with the compound of Formula I also significantly improved cardiac hypertrophy, stroke volume, cardiac output, relaxation time, dPdtₘₐₓ, end-diastolic pressure, and arterial elastance compared with vehicle treated animals. Daily treatment with the compound of Formula I did not produce a concomitant significant increase in circulating hemoglobin level or have a significant effect on other CBC parameters.

These results indicate that administration of the compound of Formula I effectively treats congestive heart failure. Administering the compound of Formula I daily for 28 days significantly improved cardiac function in congestive heart failure following MI, including significant improvement in ejection fraction, stroke work, stroke volume, cardiac output, relaxation time, cardiac hypertrophy, end-systolic volume, and cardiac contractility. Improvement in cardiac function and treatment of congestive heart failure is achieved without any concomitant rise in blood hemoglobin.

### Example 5. The Compound of Formula I, 3-[(1-cyano-4-hydroxy-7-phenoxy-isoquinoline-3-carbonyl)-amino]-3-methyl-butyric acid

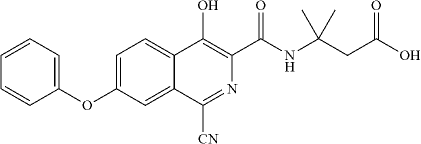 [SMILES: CC(C)(CC(=O)O)NC(=O)c1nc(C#N)c2cc(Oc3ccccc3)ccc2c1O]

The compound was made according to the methods disclosed in U.S. Pat. No. 9,409,892. In brief, to a mixture of 1-cyano-4-hydroxy-7-phenoxy-isoquinoline-3-carboxylic acid methyl ester (100 mg, 0.31 mmol) and 3-amino-3-methyl-butyric acid ((97 mg, 0.94 mmol) (Oakwood) in DMF (3 mL) was added NaOMe solid (68 mg, 1.25 mmol). The resultant mixture was heated in a 150-160° C. oil bath for 3 h. Reaction mixture was diluted with water (100 mL) and acidified by 1 N HCl to pH=3-4. Precipitate was collected and dried. Crude residue was purified by silica gel chromatography, eluting with 10-50% EtOAc/CH₂Cl₂. Fractions containing the produce were collected and concentrated. It was triturated with MeOH (2 mL). Solid was collected and dried in vacuo to provide the title compound 51 mg (0.13 mmol) in 41% yield. LC-MS ESI−: 404.06 (M−1)⁻. IC₅₀ PHD2/IC₅₀ PHD1=23.

### Example 6 Acute Cardioprotection

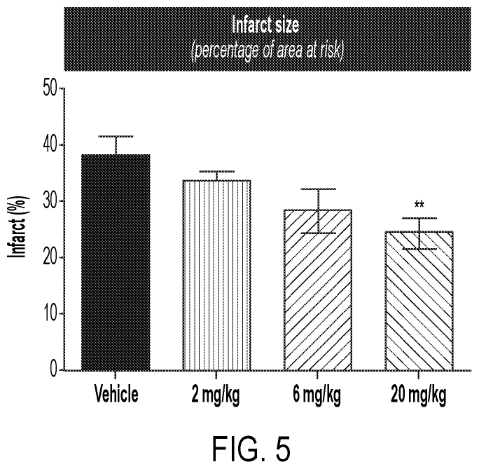

The compound of Formula I reduced infarct size post-acute myocardial ischemia in a dose-dependent manner as shown in FIG. 5.

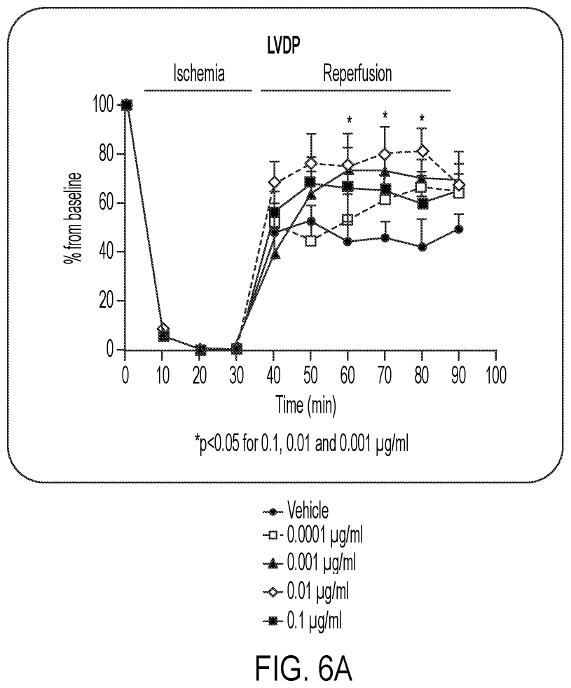

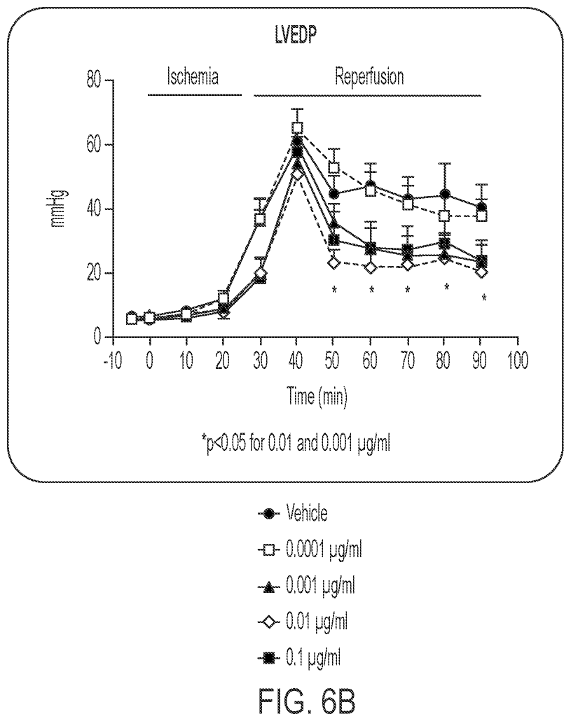

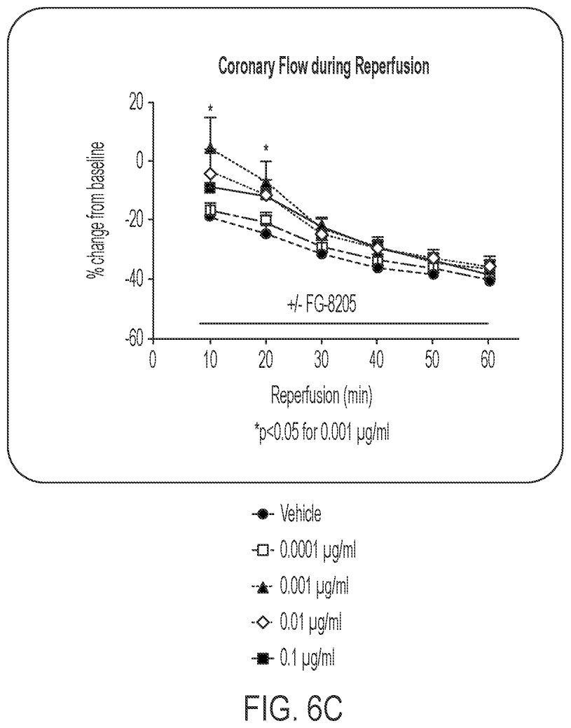

The compound of Formula I improved left ventricular function and coronary flow after global ischemia as shown in FIGS. 6A, 6B, and 6C.

### Example 7 Activity in Preclinical Heart Failure Models

Efficacy studies of the compound of Formula I showed significant post-injury improvements across multiple cardiac parameters. Experiments were carried out as described below.

A) Rat Model of Ischemic Heart Failure with Reduced Ejection Fraction (HFrEF)

### Preclinical Study Design:

Male Sprague Dawley rats
Ligation of left coronary artery for 30 minutes followed by reperfusion
Post-ligation recovery for 28 days
Cardiac function 28 days after ischemia-reperfusion using a Millar pressure-volume catheter inserted in the left ventricle via the carotid artery
Record pressure-volume loops over several cardiac cycles and derived cardiac function parameters

### Protocols and Findings:

| | Doses | Dosing | Treatment | |
|---|---|---|---|---|
| Study | (mg/kg) | Frequency | Duration | Findings |
| Acute | 6, 20 | QOD | 4 weeks | Significant dose-dependent |
| Myocardial | | | | reduction in cardiac hypertrophy |
| Ischemia | | | | and collagen deposition, |
| Induced HF | | | | improvement in systolic and |
| | | | | diastolic function (ejection fraction |
| | | | | improved at both doses). |
| Acute | 6, 20 | QOD | 4 weeks | Significant dose-dependent |
| Myocardial | | | | improved systolic function (ejection |
| Ischemia | | | | fraction improved at both doses) |
| Induced | | | | Some improvement in diastolic |
| HF/Delayed | | | | function and cardiac hypertrophy |
| Administration | | | | |
| Acute | 2, 6, 20 | Daily | 4 weeks | Significant reduction in cardiac |
| Myocardial | | | | hypertrophy and collagen |
| Ischemia | | | | deposition, improvement in systolic |
| Induced HF | | | | and diastolic function (ejection |
| | | | | fraction improved at all doses). |
| Acute | 0.2, 0.6, 2 | Daily | 4 weeks | Significant reduction in cardiac |
| Myocardial | | | | hypertrophy and improved systolic |
| Ischemia | | | | and diastolic function at 2 mg/kg |
| Induced | | | | |
| HF/MED | | | | |
| Acute | 20 | Daily | 7 days | 14 and 28 days regimens improved |
| Myocardial | | | 14 days | systolic and diastolic functions and |
| Ischemia | | | 28 days | reduced cardiac hypertrophy |
| Induced | | | | All regimens reduced collagen |
| HF/Regimes | | | | deposition |
| Permanent | 6, 20 | Daily | 4 weeks | Non-significant reduction in cardiac |
| Myocardial | | | | hypertrophy and collagen deposition |
| Ischemia | | | | at both doses |
| Induced HF | | | | No improvement in systolic or |
| | | | | diastolic function at either dose |
| Diastolic | 20 | QOD | 16 weeks | Significant improvement in |
| dysfunction | | | | diastolic function |
| HF in obese | | | | Significant stabilization of |
| ZSF1 rats | | | | hypertension |
| | | | | Improvement in plasma levels of |
| | | | | the cardiac injury marker fatty acid |
| | | | | binding protein 3 |

### Results:

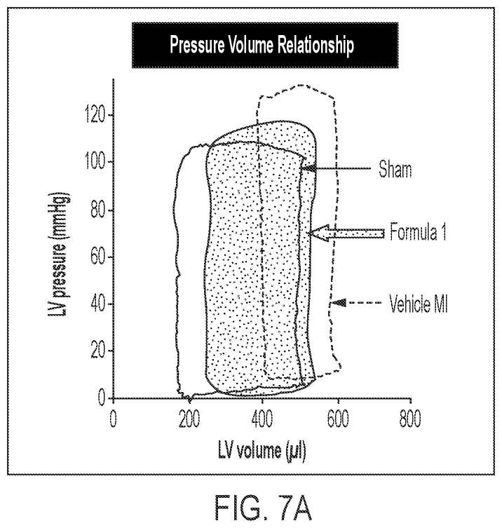

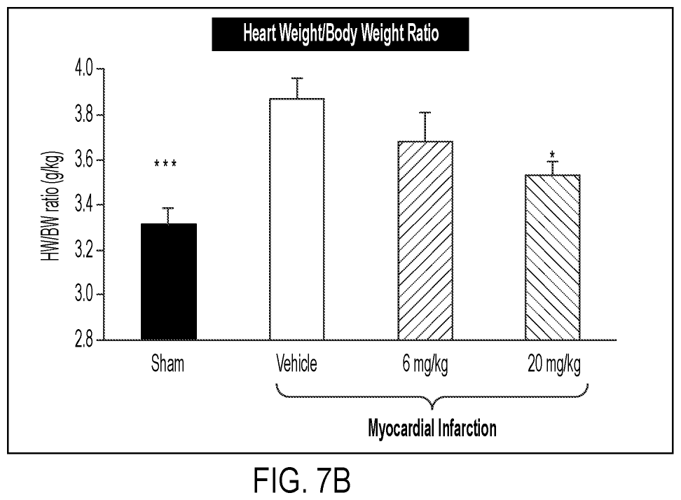

The compound of Formula I as monotherapy reduced cardiac hypertrophy as shown in FIGS. 7A and 7B, 28 days of dosing (QOD).

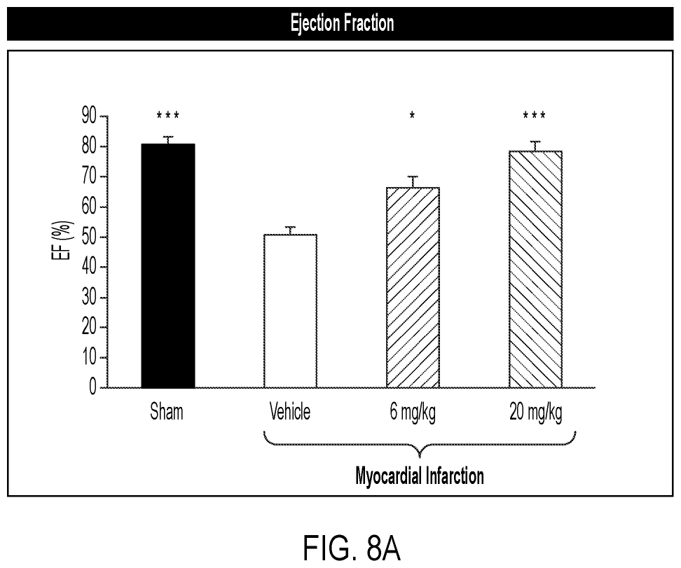

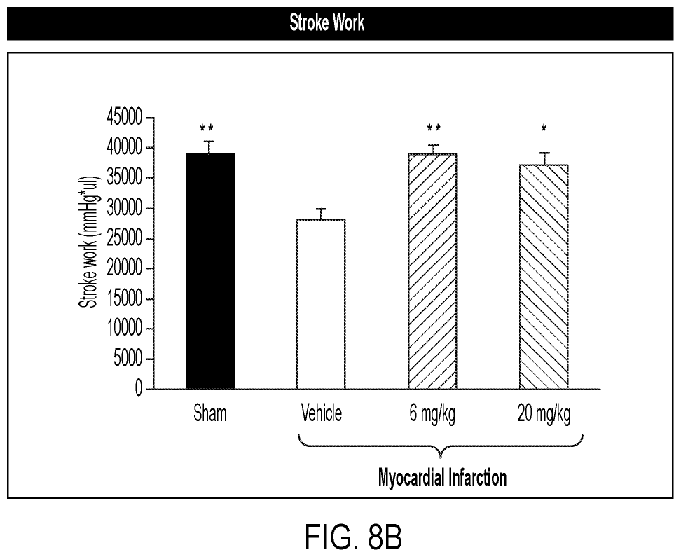

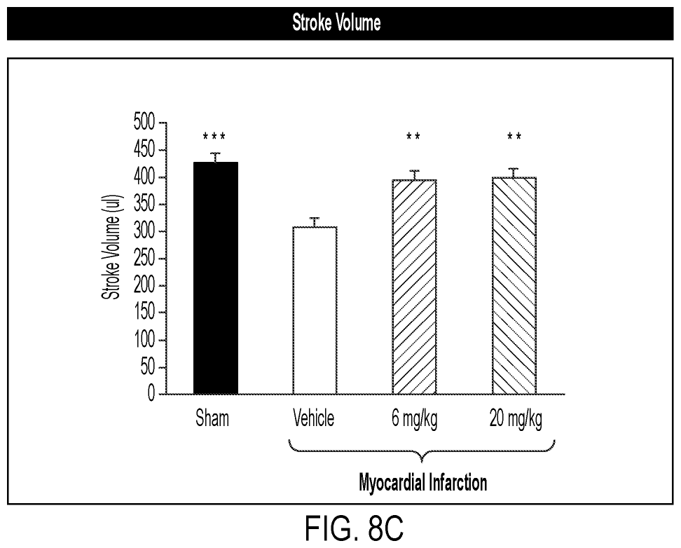

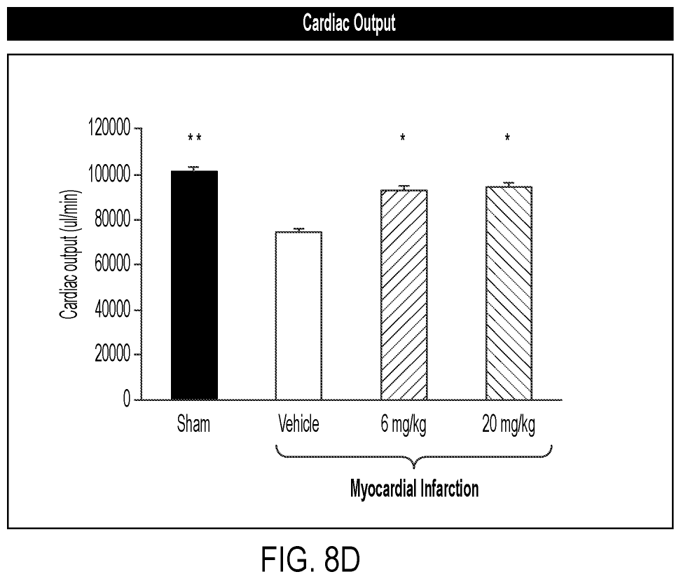

The compound of Formula I as monotherapy significantly improved cardiac function as shown in FIG. 8A ejection fraction EF, 8B stroke work, 8C stroke volume, and 8D cardiac output; 28 days of dosing (QOD).

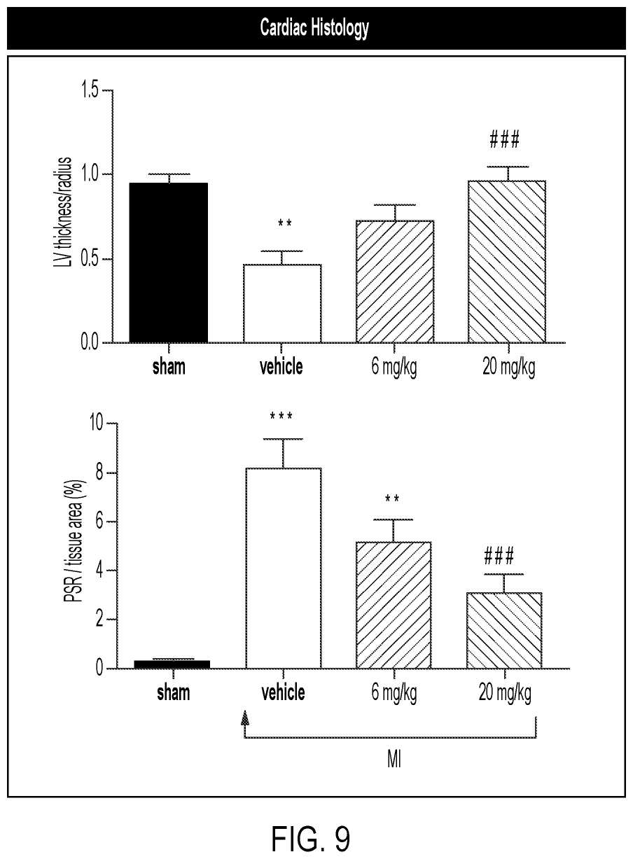

The compound of Formula I preserved wall thickness and reduced collagen deposition in the left ventricle as shown in FIG. 9, 28 days of dosing (QOD).

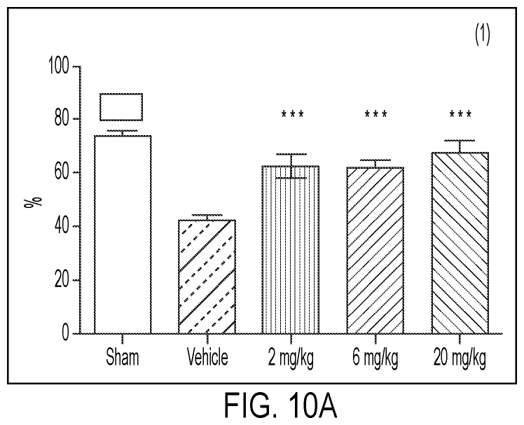

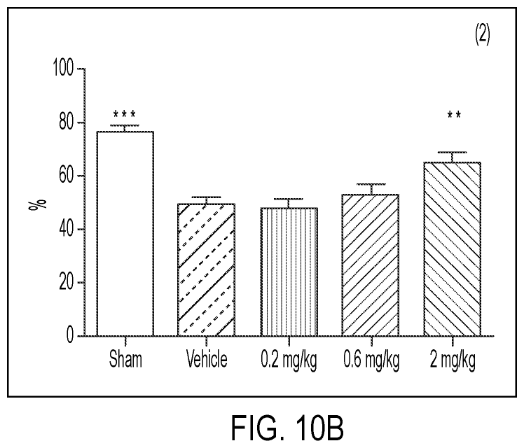

Dose response with the compound of Formula I on cardiac function in congestive heart failure (CHF) was studied by inducing CHF following myocardial ischemia for the determination of Minimum Effective Dose (MED) 2 mg/kg, as shown in FIGS. 10A and 10B.

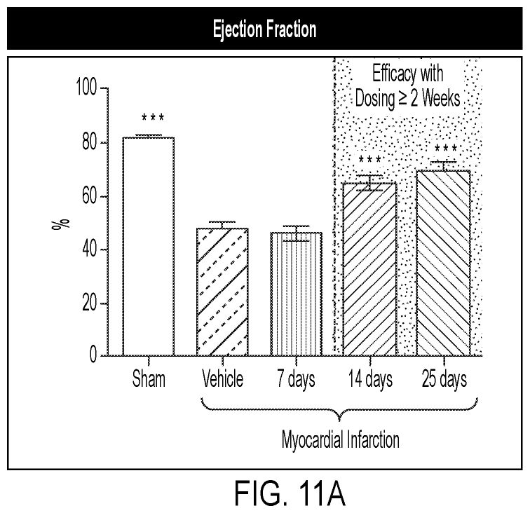

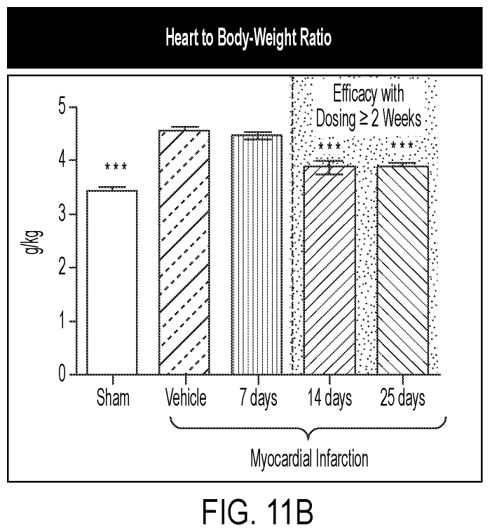

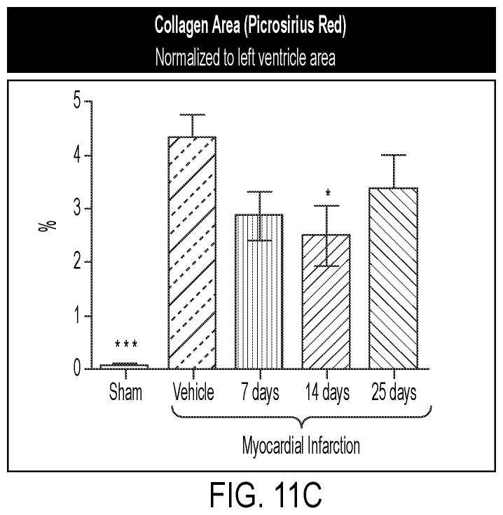

The effect of duration of administration of the compound of Formula I on cardiac function in congestive heart failure (CHF) by inducing CHF subsequent to myocardial infarction was studied to determine minimum treatment duration, as shown in FIGS. 11A, 11B and 11C.

B) ZSF1 Model of Heart Failure with Preserved Ejection Fraction (HFpEF)

This study determined the effects of the compound of Formula I on cardiac function after 16 weeks of treatment in male obese ZSF1 rats.

Lean ZSF1 rats were used as controls.

The obese ZSF1 rat was developed by crossing diabetic Zucker fatty rats with spontaneously hypertensive heart failure (SHHF) rats

### Obese ZSF1 Rats Displayed:

Hypertension
Impaired renal function
Obesity
Type 2 diabetes
Insulin resistance, hyperinsulinemia
Hypertriglyceridemia,
Hypercholesterolemia

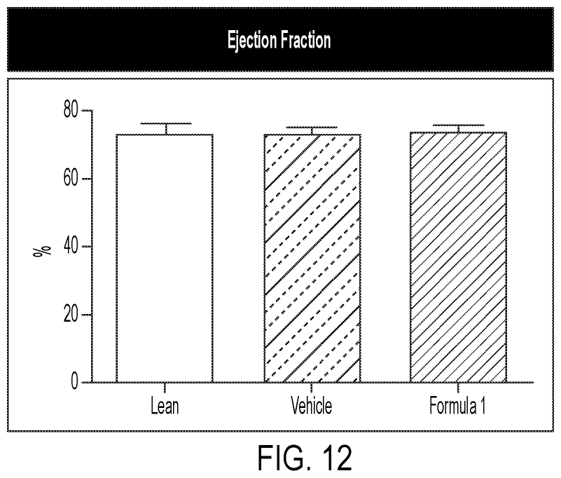

These comorbidities led to ZSF1 rats developing heart failure with a preserved ejection fraction as shown in FIG. 12.

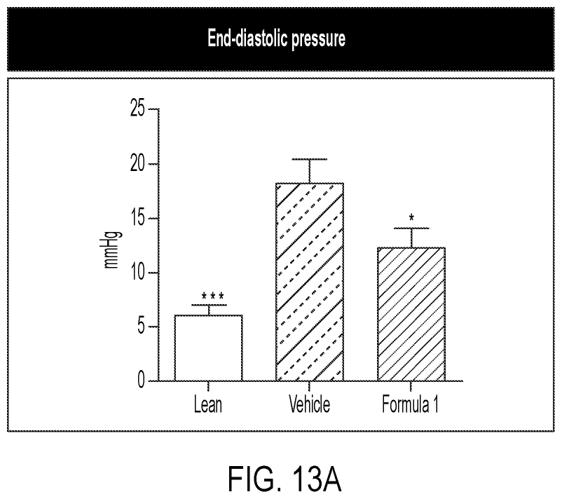

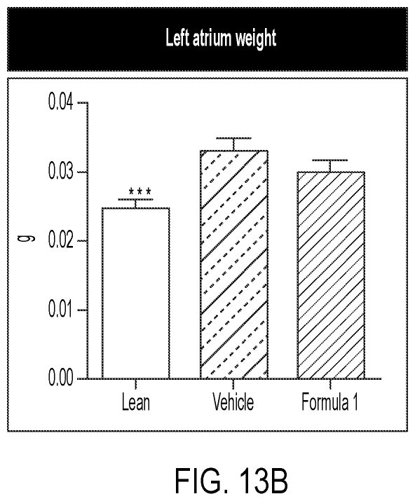

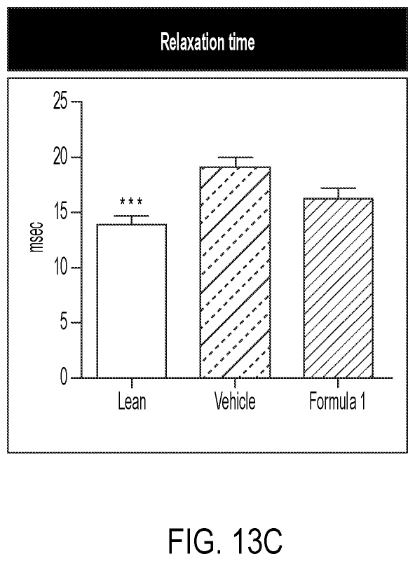

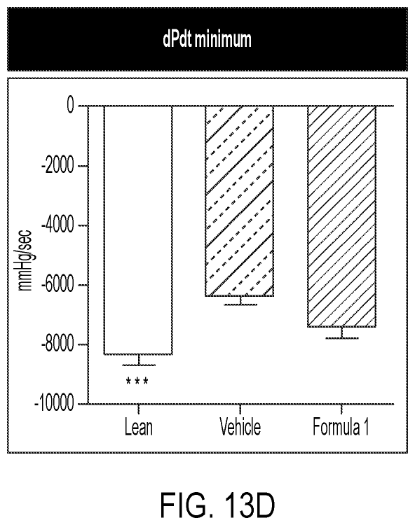

Compound of Formula I monotherapy improved diastolic function across multiple parameters as shown in FIGS. 13A end-diastolic pressure, 13B left atrium weight, 13C relaxation time, and 13D dp/dt minimum.

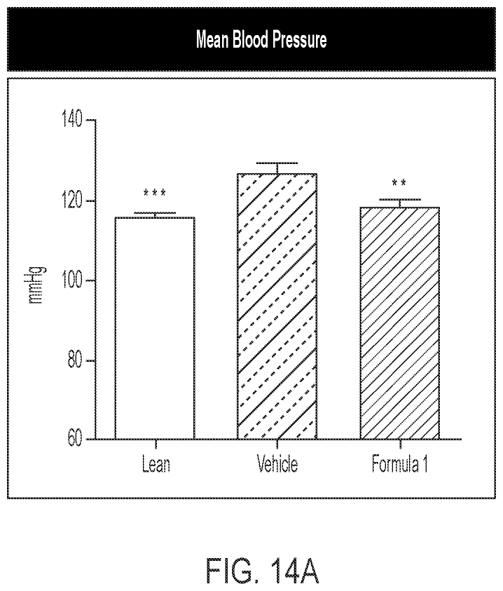

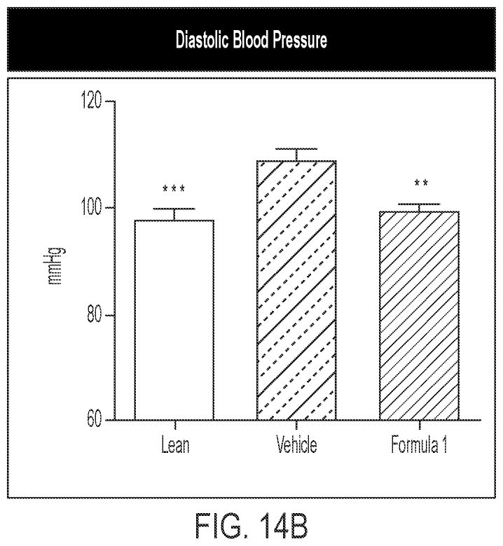

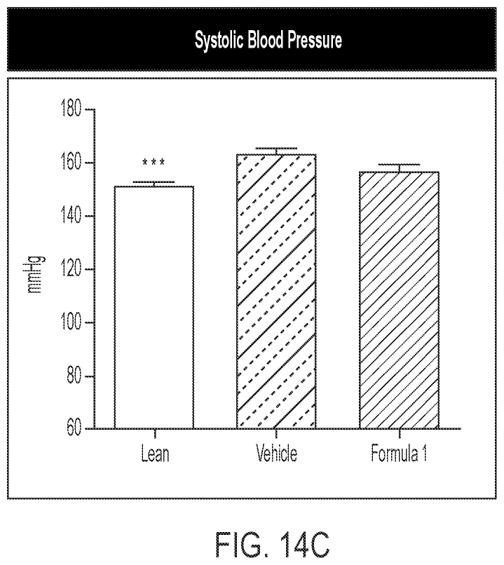

Compound of Formula I monotherapy normalized blood pressure as shown in FIGS. 14A mean blood pressure, 14B diastolic blood pressure, and 14C systolic blood pressure.

### Example 8 Compound of Formula I and SGLT2i Combination Activity in Preclinical Heart Failure Models

A) Rat Model of Ischemic Heart Failure with Reduced Ejection Fraction (HFrEF)

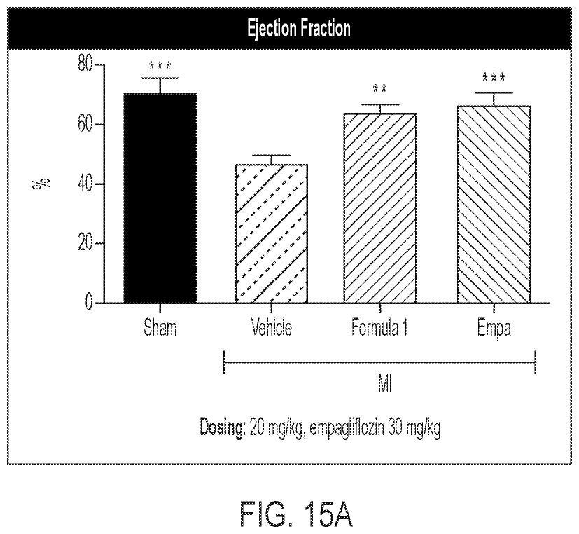

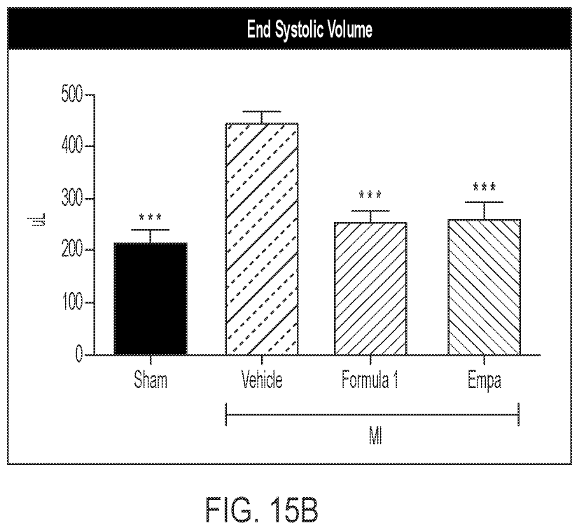

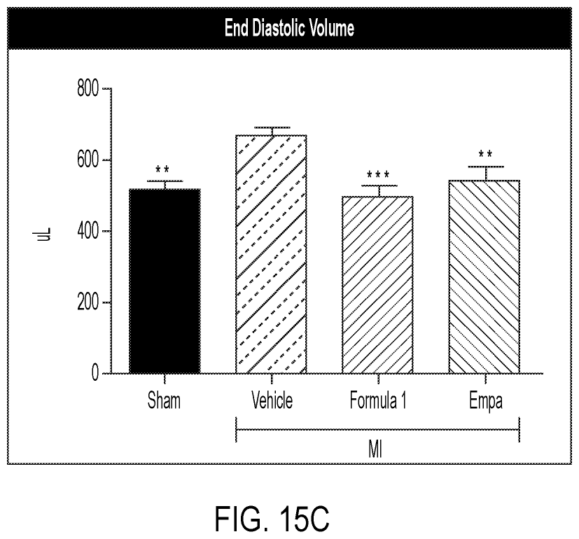

The compound of Formula I and an exemplary SGLT2i, empagliflozin, each exhibited similar efficacy as monotherapies in improving cardiac function in HFrEF as shown in FIGS. 15A ejection fraction, 15B end systolic volume, and 15C end diastolic volume.

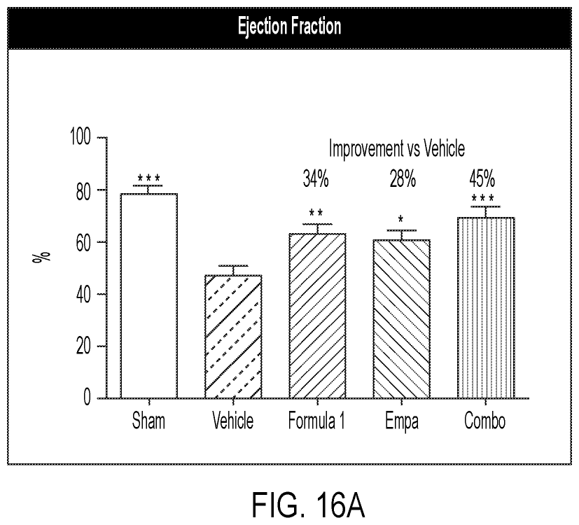

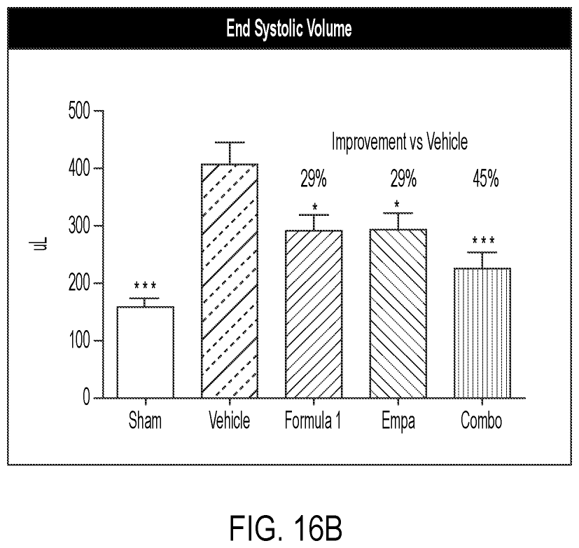

The compound of Formula I plus empagliflozin in combination showed an additive effect in the treatment of myocardial ischemia-induced heart failure as shown in FIGS. 16A and 16B.

The finding of a 40-50% improvement in ejection fraction and end systolic volume when myocardial ischemia-induced heart failure was treated with the compound of Formula I plus empagliflozin in combination demonstrated a significant additive effect of these treatments used in combination, compared to the similar ˜30% improvement observed with each treatment used as monotherapy.

B) ZSF1 Model of Heart Failure with Preserved Ejection Fraction (HFpEF)

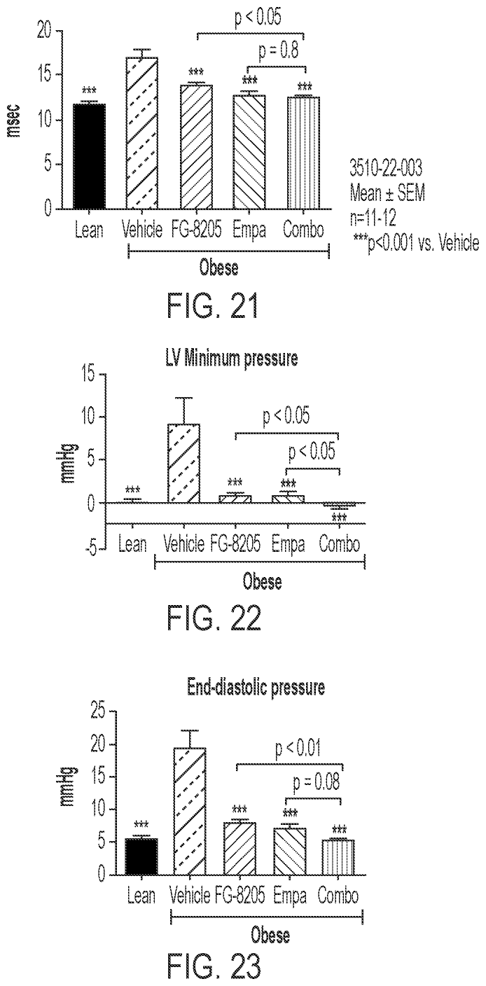

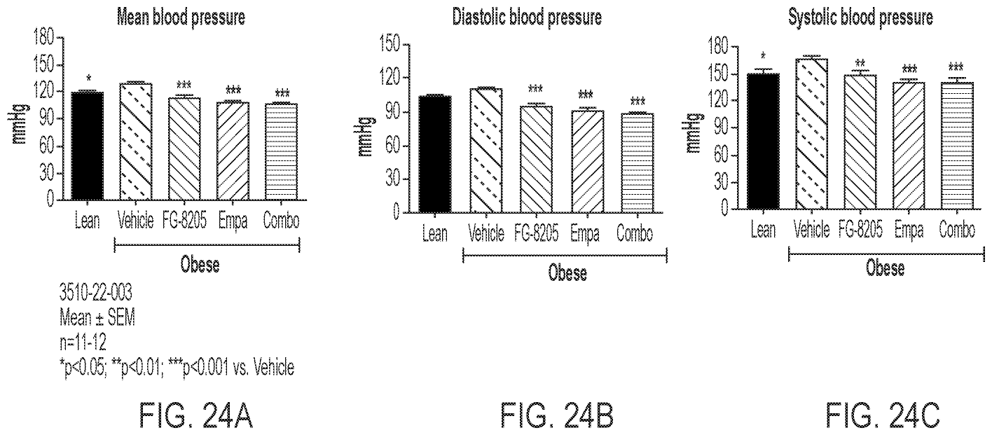

The compound of Formula I and an exemplary SGLT2i, empagliflozin, each exhibited similar efficacy as monotherapies in improving cardiac function in HFpEF as shown in FIGS. 21 (LV relaxation), 22 (LV minimum pressure), and 23 (end-diastolic pressure).

The figures also show the combination of the compound of Formula I plus empagliflozin showed an additive effect in the treatment of HFpEF as shown in Figures @@. Improvement in left ventricle filling pressure (minimum and end-diastolic pressures) was more pronounced with combination therapy than monotherapies.

### Example 9 Pharmacology

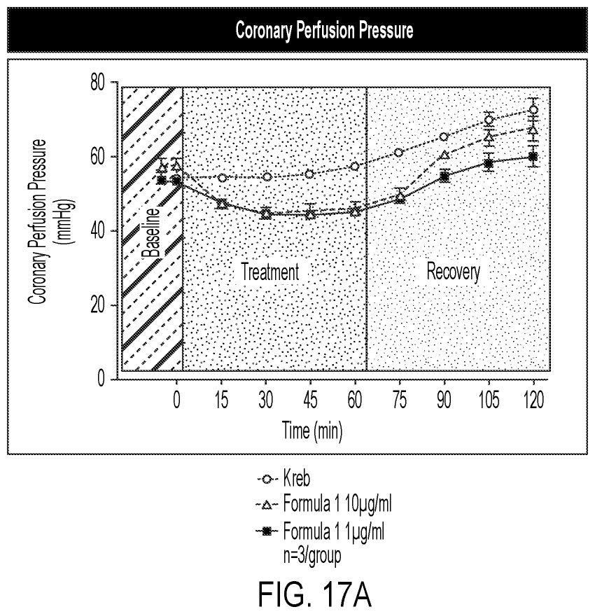

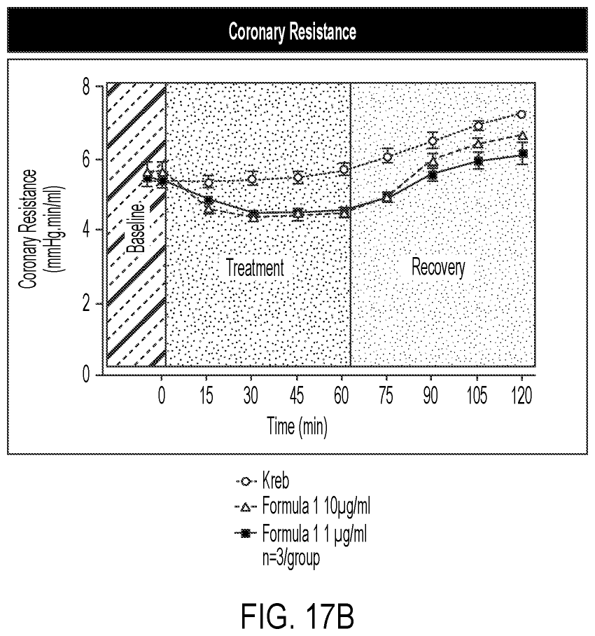

The compound of Formula I induced coronary artery vasodilation in an isolated heart preparation as shown in FIGS. 17A and 17B.

### Example 10 Pharmacokinetics (PK), Absorption, Distribution, Metabolism, and Excretion (ADME)

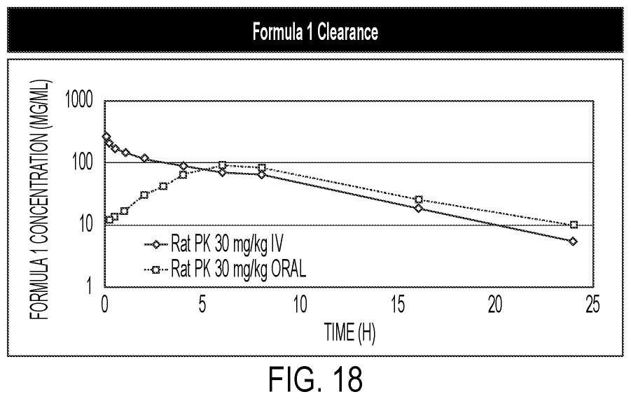

The PK profile of the Compound of Formula I in rats (high oral bioavailability & low CL) was studied and the results are shown in FIG. 18 and summarized in the following Table.

| Key PK Parameters | Mean ± SD |
|---|---|
| Tₘₐₓ (h) | 6.7 ± 1.2 |
| Cₘₐₓ (μg/mL) | 91 ± 12 |
| AUCₜ (μg * h/mL) | 1010 ± 268 |
| AUC_{inf} (μg * h/mL) | 1080 ± 328 |
| t_{1/2} (h) | 5.0 ± 1.3 |
| CL (mL/h/kg) | 22 ± 2.7 |
| Vₛₛ (mL/kg) | 123 ± 56 |

Oral bioavailability was estimated to be ˜78%
The compound of Formula I exhibited a low CL of 22 mL/h/kg, lower than hepatic blow flow rate
Vss was higher than plasma volume, suggesting possible distribution in tissue compartments

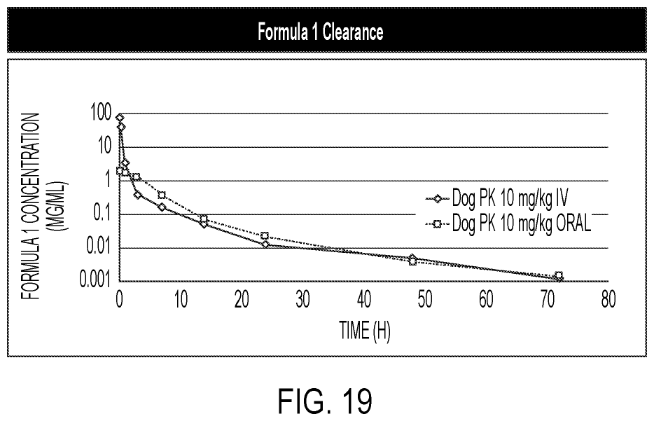

The preliminary PK profile of the compound of Formula I in dogs was studied and the results are shown in FIG. 19 and summarized in the following Table.

| Key PK Parameters | Mean ± SD |
|---|---|
| Tmax (h) | 0.8 ± 0.4 |
| Cmax (μg/mL) | 2.6 ± 1.9 |
| AUCₜ (μg * h/mL) | 10.2 ± 3.3 |
| AUC_{inf} (μg * h/mL) | 10.2 ± 3.3 |
| t_{1/2} (h) | 11.3 ± 1.8 |
| CL (mL/h/kg) | 252 ± 41 |
| Vₛₛ (mL/kg) | 249 ± 73 |

Oral bioavailability was estimated to be ˜25% in dogs
Clearance in dogs was higher than in rats (approx 5-10× of CL in rats)
Vss was higher than plasma volume, suggesting possible distribution in tissue compartments

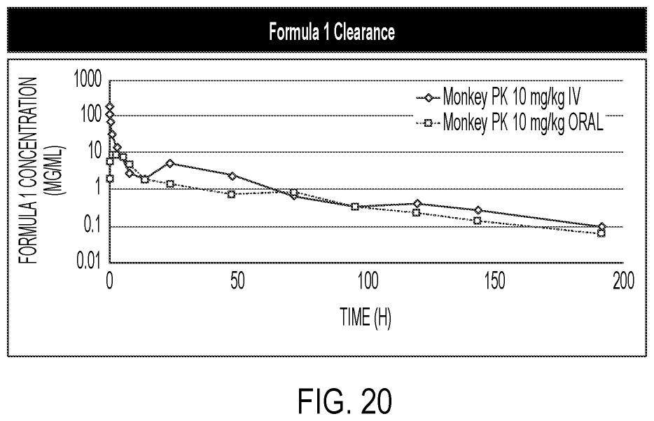

The PK profile of the compound of Formula I in monkeys was studied and the results are shown in FIG. 20 and summarized in the following Table. Notably, the compound was found to be of good oral bioavailability.

| Key PK Parameters | |
|---|---|
| (males and females) | Mean ± SD |
| Tₘₐₓ (h) | 2.6 ± 1.6 |
| Cₘₐₓ (μg/mL) | 10.9 ± 3.7 |
| AUCₜ (μg * h/mL) | 176 ± 67 |
| AUC_{inf} (μg * h/mL) | 180 ± 68 |
| t_{1/2} (h) | 34.6 ± 9.5 |
| CL (mL/h/kg) | 26 ± 11 |
| Vₛₛ (mL/kg) | 184 ± 28 |

Oral bioavailability ranged from 36 to 48% (group mean of 42-47%)
Generally, there was no marked sex difference in compound exposure or key PK parameters
Compound exposure increased dose proportionally
Systemic clearance in monkey was comparable to those in rats (but slower than CL in dogs)
Vss was higher than plasma volume, suggesting possible distribution in tissue compartments

PK characteristics of the compound of Formula I are summarized in the following Table.

| Parameters | Rat | Dog | Monkey |
|---|---|---|---|
| Oral % F | 78% | 25% | 36 to 71% |
| T_{1/2} (h) | Variable: | 11 ± 2 | 22 to 36 |
| | Mostly 5-6 h, | | |
| | some longer | | |
| | (10-15 h) | | |
| Tmax (h) | 5-6 hr | Mostly 1 hr | Mostly 1-hr |
| CL (mL/kg/hr) | 22 to 40 | 252 ± 41 | 31 to 40 |
| Dose | Yes | Yes | Yes |
| proportional | (10-150 mg/kg) | (10-60 mg/kg) | (10-60 mg/kg) |
| | | less than dose | less than dose |
| | | prop (up | prop (up |
| | | to 150 mg/kg) | to 150 mg/kg) |
| Accumulation | Unclear | (No data, | 2-3 fold higher |
| (after multiple | (once daily | but expect | compared to |
| dosing) | dosing 2 weeks) | some acc based | Day 1 exposure |
| | Yes (4 wk | on T_{1/2}) | (consistent |
| | GLP TK) | | with T_{1/2} and |
| | | | daily dosing) |
| Sex difference | No | No | No |
| in PK | | | |

The distribution of the compound of Formula I in tissues of interest is summarized in the following Table.

| | 4 h post dose | | 8 h post dose | |
|---|---|---|---|---|
| | Compound of | Tissue/ | Compound of | Tissue/ |
| | Formula 1 | plasma | Formula 1 | plasma |
| Tissue | (μg/mL or μg/g) | ratio | (μg/mL or μg/g) | ratio |
| Plasma | 56.6 ± 10.6 | 1.00 | 43.3 ± 11.0 | 1.00 |
| Colon | 12.9 ± 2.7 | 0.23 | 16.1 ± 6.3 | 0.37 |
| Duodenum | 19.1 ± 5.6 | 0.34 | 21.8 ± 4.1 | 0.50 |
| Heart | 10.4 ± 4.6 | 0.18 | 7.4 ± 2.1 | 0.17 |
| Kidney | 9.2 ± 3.4 | 0.16 | 7.2 ± 1.9 | 0.17 |
| Liver | 40.2 ± 7.2 | 0.71 | 38.4 ± 4.6 | 0.89 |

N=3/time point, male Swiss Webster mice, single iv dose, 60 mg/kg Compound of Formula I

All references cited herein are hereby incorporated by reference herein in their entirety.

What is claimed is:

1. A method for treating congestive heart failure in a subject in need thereof, said method comprising administering to the subject an effective amount of the compound of Formula I 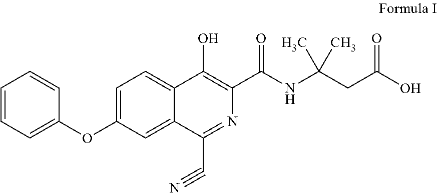 [SMILES: CC(C)(CC(=O)O)NC(=O)c1nc(C#N)c2cc(Oc3ccccc3)ccc2c1O]
thereby treating the congestive heart failure.

2. A method for improving cardiac function in a subject in need thereof, said method comprising administering to said subject an effective amount of the compound of Formula I 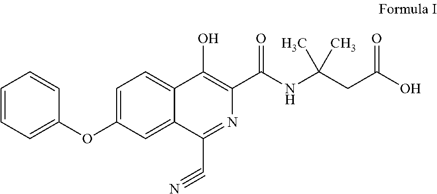 [SMILES: CC(C)(CC(=O)O)NC(=O)c1nc(C#N)c2cc(Oc3ccccc3)ccc2c1O]
thereby improving cardiac function.

3. The method of claim 2, wherein the cardiac function is selected from the group consisting of ejection fraction, stroke work, stroke volume, cardiac output, relaxation time, end systolic volume, end diastolic volume, and cardiac contractility.

4. A method for preventing or reducing decline in cardiac function in a subject in need thereof, said method comprising administering to said subject an effective amount of the compound of Formula I 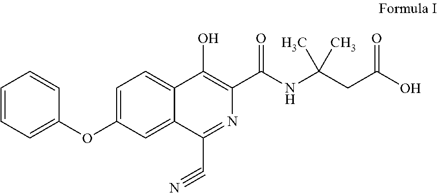 [SMILES: CC(C)(CC(=O)O)NC(=O)c1nc(C#N)c2cc(Oc3ccccc3)ccc2c1O]
thereby preventing or reducing the decline in cardiac function.

5. The method of claim 4, wherein the cardiac function is selected from the group consisting of ejection fraction, stroke work, stroke volume, cardiac output, relaxation time, end systolic volume, end diastolic volume, and cardiac contractility.

6. A method for reducing cardiac damage in a subject in need thereof, said method comprising administering to said subject an effective amount of the compound of Formula I 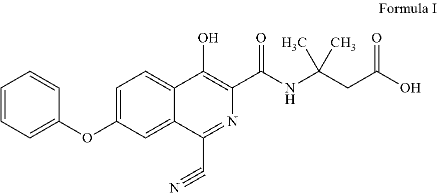 [SMILES: CC(C)(CC(=O)O)NC(=O)c1nc(C#N)c2cc(Oc3ccccc3)ccc2c1O]
thereby reducing cardiac damage.

7. The method of claim 6, wherein the cardiac damage is selected from the group consisting of infarct size, cardiac hypertrophy, and peri-infarct vessel formation.

8. The method of any one of claims 1-7, wherein the subject has or is at risk for having congestive heart failure.

9. The method of any one of claims 1-8, wherein the subject has been diagnosed with a disorder selected from the group consisting of a myocardial infarction, atherosclerosis, cardiomyopathy, congenital heart disease, and rheumatic fever.

10. The method of any one of claims 1-9, wherein the administering comprises periodic administration of the compound over an extended duration of treatment.

11. The method of claim 10, wherein the frequency of administration is selected from the group comprising once every 12 hours, once every 24 hours, once every 48 hours, once every 72 hours, once every day, once every 2 days, once every 3 days, once every 4 days, once every 5 days, once every 6 days, and once every week.

12. The method of any one of claims 1-11, wherein the method does not produce a concomitant significant increase in circulating hemoglobin levels in the subject.

13. The method of any one of claims 1-12, wherein the compound of Formula I is used in combination with a sodium-glucose cotransporter 2 (SGLT2) inhibitor.

14. The method of claim 13, wherein the SGLT2 inhibitor is selected from the group consisting of canagliflozin, dapagliflozin, and empagliflozin.
